(19) United States
(12) Patent Application Publication
(10) Pub. No.: US 2015/0148405 A1
(43) Pub. Date: May 28, 2015

(54) Expression Construct for a Lin28-Resistant Let-7 Precursor MicroRNA

(71) Applicant: THE JOHNS HOPKINS UNIVERSITY, BALTIMORE, MD (US)

(72) Inventors: Mollie Katherine Meffert, Baltimore, MD (US); Claudia R. Ruiz-Garzon, Baltimore, MD (US); Yu-Wen A. Huang, Baltimore, MD (US); Elizabeth C. H. Eyler, Baltimore, MD (US)

(73) Assignee: THE JOHNS HOPKINS UNIVERSITY, BALTIMORE, MD (US)

(21) Appl. No.: 14/380,279

(22) PCT Filed: Feb. 21, 2013
(86) PCT No.: PCT/US2013/027187

§ 371 Date: Aug. 21, 2014

Related U.S. Application Data
(60) Provisional application No. 61/601,230, filed on Feb. 21, 2012.

(51) Int. Cl. C12N 15/113; C12N 15/10

(52) U.S. Cl. CPC C12N 15/113; C12N 15/102; C12N 2330/50; C12N 2320/51; C12N 2310/141; C12N 2310/531
USPC 514/44 R; 536/23.1; 435/320.1; 435/366; 435/442

(57) ABSTRACT

Precursor microRNA molecules that have been modified to prevent a protein involved in regulating developmental timing, oncogenesis, and/or neuronal growth from blocking processing of the precursor microRNA sequence to the mature microRNA are provided. Methods and kits for using the precursor microRNA molecules also are provided.

### CROSS-REFERENCE TO RELATED APPLICATIONS

This application claims the benefit of U.S. Provisional Application No. 61/601,230, filed Feb. 21, 2012, which is incorporated herein by reference in its entirety.

### BACKGROUND

The control of gene expression at the level of translation is vital to neuronal function and synaptic plasticity. Dysregulated translation has been linked to cognitive disorders, including Fragile X syndrome, Autism, and Parkinson's disease. The regulation of translation plays a key role in the neuronal response to multiple stimuli, including synaptic activity (Huber et al., 2000; Raab-Graham et al., 2006; Wang et al., 2009), depolarization (Schratt et al., 2004), retinoic acid (Aoto et al., 2008), and neurotrophins (Aakalu et al., 2001; Jaworski et al., 2005; Schratt et al., 2004). While most of these stimuli enhance total cellular protein synthesis, their responses demonstrate marked transcript specificity. This has been best defined for the brain-derived neurotrophic factor (BDNF), which is broadly expressed in the mammalian brain and plays pivotal roles in neuronal survival, structure, and synapse function. The effects of BDNF on protein synthesis, while physiologically important, are quite selective with an estimated 4% or less of expressed mRNAs undergoing enhanced translation (Schratt et al., 2004; Yin et al., 2002) despite a general enhancement of cap-dependent initiation and elongation by BDNF (Takei et al., 2009).

Mechanisms conferring specificity to post-transcriptional control of gene expression are incompletely defined. mRNA regulatory elements and binding proteins provide significant examples of control for specific transcripts, but explanations for concerted changes in groups of mRNAs are largely lacking. While subcellular restriction of stimulus-dependent signals in neurons likely imparts some transcript selectivity, target specificity remains inadequately explained since hundreds of mRNAs populate discrete cellular compartments, such as dendrites.

### SUMMARY

The presently disclosed subject matter provides precursor microRNA molecules that have been modified to prevent a protein involved in regulating developmental timing, oncogenesis, and/or neuronal growth from blocking processing of the precursor microRNA sequence to the mature microRNA. More particularly, in one aspect, the presently disclosed subject matter provides a precursor microRNA molecule, the precursor microRNA molecule comprising a nucleic acid comprising: (a) a precursor microRNA sequence; and (b) a mutation in the precursor microRNA sequence that prevents a protein involved in regulating developmental timing, oncogenesis, and/or neuronal growth from blocking processing of the precursor microRNA sequence to a mature microRNA.

In another aspect, the presently disclosed subject matter provides DNA molecules encoding the precursor microRNA molecule of the presently disclosed subject matter.

In yet another aspect, kits comprising a DNA molecule encoding the precursor microRNA molecule of the presently disclosed subject matter are provided.

In a further aspect, cells comprising DNA molecules and/or the precursor microRNA molecules of the presently disclosed subject matter are provided. Accordingly, in some aspects, the presently disclosed precursor microRNA sequence can be inserted into a viral backbone, thereby allowing production of virus encoding the precurosor miRNA and an efficient means (e.g., by viral infection) to introduce the precurosor miRNA for cellular expression.

In another aspect, the presently disclosed subject matter provides a method for blocking the ability of a protein involved in regulating developmental timing, oncogenesis, and/or neuronal growth to decrease a level of a microRNA molecule, the method comprising: (a) providing a microRNA sequence; and (b) making a mutation in the microRNA sequence such that the ability of a protein involved in regulating developmental timing, oncogenesis, and/or neuronal growth to decrease the levels of a microRNA molecule is blocked.

In still another aspect, the presently disclosed subject matter provides a method for elevating levels of a microRNA in a cell, the method comprising: (a) providing a microRNA sequence; (b) making a mutation in the microRNA sequence such that the ability of a protein involved in regulating developmental timing, oncogenesis, and/or neuronal growth to decrease a level of a microRNA molecule is blocked; and (c) expressing the microRNA sequence in a cell.

Certain aspects of the presently disclosed subject matter having been stated hereinabove, which are addressed in whole or in part by the presently disclosed subject matter, other aspects will become evident as the description proceeds when taken in connection with the accompanying Examples and Figures as best described herein below.

### BRIEF DESCRIPTION OF THE FIGURES

Having thus described the presently disclosed subject matter in general terms, reference will now be made to the accompanying Figures, which are not necessarily drawn to scale, and wherein:

FIGS. 1A-1G show that BDNF increases P-body formation in soma and dendrites of hippocampal neurons: (A) Endogenous GW182 (top panel) colocalizes with GFP-Dcp1a (middle panel) in neuronal dendrites; overlay (bottom panel); (B) P-body formation in dendrites of hippocampal pyramidal neurons following mock- (top panels) or BDNF-stimulation (bottom panels, 100 ng/mL); (C) P-body formation in cell somas following mock- (top panels) or BDNF-stimulation (bottom panels), t=min post-stimulation; (D) Quantification and time course of percent change in GFP-Dcp1a P-body numbers in neuronal dendrites following mock- (open circles) or BDNF-stimulation (closed circles) in the presence of Actinomycin D (0.5 μg/mL) to isolate changes due to translation, *=p<0.05 by one-way ANOVA with Bonferroni correction; (E) Lysates from mock (−) or BDNF (+, 1 hour) stimulated neuronal cultures immunoprecipitated (IP) with GW182 antiserum (IP-GW) or isotype-control serum (IP-Ctrl). Input is 20% of IP'd protein; (F) Densitometric quantification from 9 independent experiments, as in (E); mock condition (open bars) set as 1.0; and (G) Total RNA, measured by A260, recovered by GW182 IP from equal lysate inputs; mock (open bar) set as 1.0. BDNF increased GW182-associated RNA 2.62±0.29 fold. All error bars represent SEM. *=p<0.05 by unpaired Student's t-test. Scale bars represent 10 μm. See also FIG. 8;

FIGS. 2A-2D show that miRNA-mediated repression is enhanced by BDNF and associated with BDNF target specificity: (A) Loss of GW182 function by shRNA targeting GW182 (GW182KD) or GFP-DNGW182 expression does not alter BDNF-enhancement of aggregate protein synthesis relative to control (uninfected) cells, or cells expressing scrambled GW182 shRNA or GFP alone. Total protein synthesis was monitored under mock (open bars) or BDNF (hatched bars, 100 ng/mL, 2 hr) stimulated conditions, and plotted relative to the control mock condition set as 1.0 (first open bar). A translation inhibitor (rapamycin, 20 μg/mL) demonstrates that observed changes are due to translation; (B) Left: Immunoblotting for BDNF target proteins in neurons either uninfected or infected with lentivirus expressing GW182 shRNA (GW182KD) or a mismatched control shRNA (sh-Control-1). mCherry is co-expressed from the virus. Right: Protein levels, normalized to 3-tubulin, of representative BDNF-upregulated targets under mock- (open bars) or BDNF-(hatched bars, 100 ng/mL, 2 hr) stimulation in the presence or absence of GW182KD (control mock, white bars, set as 1.0); n=6 independent experiments; (C) Left: Immunoblotting for BDNF target proteins in neurons either uninfected or infected with lentivirus expressing GFP-DNGW182 or GFP. Right: Protein levels, normalized to β-tubulin, of representative BDNF up- or down-regulated targets under mock (open bars) and BDNF (hatched bars) stimulated conditions in cells expressing GFP-DNGW182, GFP, or control uninfected cells (control mock, white bars, set as 1.0); n=6 independent experiments; (D) miRNA function is inhibited by GW182KD and GFP-DNGW182 but not by knockdown of LSm5 (LSm5KD). Left: luciferase activities of siRNA- or miRNA-reporter constructs in cells expressing reporter alone (−sh-CXCR4), or co-expressing reporter and CXCR4 shRNA (+sh-CXCR4), with or without GW182KD, GFP-DNGW182, or LSm5KD. Normalized luciferase values are shown relative to levels without sh-CXCR4 (set as 1.0). Right: Diagram of reporter constructs; (E) LSm5 knockdown did not alter protein synthesis of representative BDNF targets. Left Immunoblotting for BDNF targets in neurons expressing control shRNA (shControl-2) or shRNA against LSmS (LSm5KD) following mock (−) or BDNF (+) stimulation (100 ng/mL, 2 hr). Right: Densitometric quantification of 3 independent immunoblots, normalized to GAPDH and plotted relative to mock-stimulated controls (sh-Control-2-mock); (F) BDNF enhances repression of a miRNA-reporter by a small RNA hairpin (sh-CXCR4). Normalized luciferase values are shown for mock (open bars) or BDNF-stimulated (hatched bars) neurons co-expressing the miRNA reporter and either sh-Control-2 or a dose-titration of sh-CXCR4. Low-dose sh-CXCR4 repressed the miRNA-reporter in BDNF- but not mock-stimulated conditions. All experiments done in the presence of Actinomycin-D. Error bars represent SEM. *=p<0.05 in comparison to reporter alone condition (− sh-CXCR4, D and F) or mock (open bars) by unpaired Student's t-test. See also FIGS. 9-11;

FIGS. 3A-3G show that BDNF increases Dicer levels and the biogenesis of mature miRNAs: (A) Left: percentage of miRNAs from Taqman miRNA array with levels decreased over 50% (open bar) or increased over 2-fold (black bar) by BDNF (30 min., plus Actinomycin-D) Right: scatter plot of relative quantities (RQ) of individual miRNA species (solid circles) following BDNF relative to mock-stimulation. Solid line=1.0 or no change; each dot above the line represents a miRNA species increased by BDNF, each dot below the line represents a miRNA species decreased by BDNF. Normalization is to averaged reference RNAs U6snRNA, and snoRNA202, which are unchanged by BDNF; n=3 separate miRNA array pairs for mock and BDNF conditions; (B) Immunoblot of cultured hippocampal neurons stimulated with BDNF for indicated min in the presence of Actinomycin-D. Dicer peaks near 20 and declines by 60 min; (C) Top panel, BDNF enhances TRBP and ERK phosphorylation as shown by immunoblot for TRBP and phospho-Erk. Cultured hippocampal neurons were stimulated with BDNF for indicated min in the presence of Actinomycin-D. Bottom panel, lysates incubated with A-phosphatase (A-phos) as indicated demonstrate loss of phosphorylated TRBP (upper band); (D) P-body appearance in dendrites of hippocampal pyramidal neurons expressing control (sh-control-2, top panels) or Dicer-targeting shRNA (DicerKD, bottom panels) following BDNF, t=min post-stimulation; (E) Quantification and time course of P-body numbers in Dicer-deficient (DicerKD, boxes) or control (sh-control-2, circles) expressing hippocampal neurons following mock- (open shapes) or BDNF-stimulation (closed shapes); (F) Quantification and time course of P-body numbers in hippocampal pyramidal neurons treated with enoxacin (15 μM, closed circles) or oxolinic acid (15 μM, open circles); and (G) The effect of Dicer loss on BDNF-regulated protein synthesis. Top panel, immunoblotting for BDNF target proteins in Dicer-wildtype (Dicer^{flox/flox}) or Dicer deficient (Dicer^{-/-}) neurons. Cre^{ERT2}-expressing cells were treated with 4-hydroxy tamoxifen (800 nM) to induce recombination for 2.5 days before BDNF stimulation. Asterisk indicates non-specific band. Bottom panel, densitometric quantification of immunoblots. All error bars represent SEM. *=p<0.05 by unpaired Student's t-test. See also FIG. 11.

FIGS. 4A-4E show that BDNF induces Lin28, selectively diminishes Lin28-regulated miRNAs, and specifically upregulates a heterologous reporter containing Let-7 binding sites: (A) Lin28a (top panel) and Lin28b (lower panel) immunoblots of lysates from cultured hippocampal neurons stimulated with BDNF for indicated min; (B) Timecourse of BDNF-induced reductions in Lin28-regulated miRNA levels by individual TaqMan RT-qPCR reactions in mock- (BDNF 0′) or BDNF-stimulated neurons. miRNA levels were normalized to 18s rRNA and plotted relative to each mock-stimulated condition (set as 1.0). All samples underwent equal duration Actinomycin-D incubation prior to harvest; (C) Northern blot (left) and quantitation (right) of pre- and mature miRNA levels of a Lin28-target (Let-7a) or control miRNA (miR-17) in mock or BDNF-treated (30 min) neurons; (D) A binding site for Let-7 miRNAs in the 3′UTR of an mRNA confers upregulation of protein synthesis in response to BDNF. Neurons expressing Let-7 reporters containing two functional (Let-7 WT) or mutated (Let-7 Mut) Let-7 miRNA binding sites in the 3′UTR of firefly luciferase, or a reporter lacking miRNA binding sites were mock- or BDNF-stimulated (4 hr). Luciferase activities are normalized to co-expressed constitutive p-galactosidase activity and plotted relative to mock-stimulation for each reporter. All error bars represent SEM. *=p<0.05 by unpaired Student's t-test; and (E) Predicted binding sites for Lin28-targeted miRNA. The presence of a Lin28-targeted miRNA binding site in the 3′UTR of transcripts for which translation is BDNF-upregulated, BDNF-downregulated (dark gray), and BDNF-nonregulated (black) as predicted by TargetScan, PITA, Pictar, MiRanda, and miRwalk. Pink boxes denote the presence of a miRNA binding site in which the miRNA seed sequence (nucleotides 2-7) paired as a perfect or G-U wobble-containing match. Light gray boxes denote the absence of a miRNA binding site. See also FIG. 12;

FIGS. 5A-5D show that Lin28 is required for relief of miRNA-mediated repression and selective induction of BDNF-upregulated mRNA targets: (A) Loss of Lin28 prevents BDNF-induced decreases in mature Let-7a levels. Mature Let-7a levels were assessed by RT-qPCR from neurons infected with lentivirus expressing either control shRNA (sh-control-2) or shRNA targeting Lin28a (Lin28aKD) and mock- or BDNF-stimulated for 20 min (no Actinomycin-D); normalization was to control mock values (open bar, set as 1.0, n=3); (B) Effect of Lin28a loss on BDNF-regulated protein synthesis Immunoblotting of BDNF targets in control or Lin28a-deficient cells, mock- or BDNF-stimulated (left panel). Densitometric quantification of protein levels (right, top panel, n=6 each condition). Total mRNA levels for both BDNF-upregulated or downregulated targets (right, bottom panel); (C) Effect of Lin28a KD on BDNF-induced association of protein and RNA P-body components. Lysates were immunoprecipitated with anti-GW182 antibody in control (sh-Control-2) or Lin28adeficient cells, mock- or BDNF-stimulated Immunoblotting for co-IP'd Ago2 and Dcp1a (left panel) and densitometric quantification (right, top panel, n=3). Total RNA from GW182 IP of equal lysate inputs from Lin28a knockdown (Lin28aKD) or control (sh-Control-2) neurons; mock (open bars) set as 1.0. BDNF-induced increase in GW182-associated total RNA remains intact after Lin28a knockdown (right, bottom panel); and (D) Abundance of BDNF mRNA targets associated with GW182 in control (sh-Control-2) or Lin28a-deficient cells. In Lin28a-deficient neurons, mRNAs for BDNF-upregulated targets remain associated with GW182 in the presence of BDNF while the response of mRNAs for BDNF-downregulated targets is unchanged. 18s rRNA is nondetectable, ND. Error bars represent SEM. *=p<0.05 Student's t-test;

FIGS. 6A-6D show that Lin28-mediated degradation of Let-7 precursors is required for induction of BDNF-upregulated targets and neuronal outgrowth: (A) Lentiviral-mediated expression of wild-type (Let-7^{WT}) or Lin28-resistant (Let-7^{LR}) Let-7 pre-miRNAs in neurons produced dose-dependent enhancement of mature Let-7a miRNA levels assessed by RT-qPCR and shown as fold change relative to infection with virus expressing GFP alone (gray bar, set as 1.0); 1× or 2× refers to viral dose; (B) Expression of Let-7^{LR}, but not Let-7^{WT}, blocks specificity of BDNF for upregulated targets. Reporter assays in mock- (open bars or BDNF-stimulated (hatched bars) neurons with luciferase constructs fused to the 3′UTR from BDNF-upregulated targets (GluR1 or CaMKIIu) or a downregulated target (KCC2); (C) BDNF-induced dendrite outgrowth requires Lin28-mediated degradation of miRNA precursors. Dendrite complexity is quantitated for neurons expressing Let-7^{WT} (black circles) or Let-7^{LR} (gray triangles) following mock- (open shapes) or BDNF- (25 ng/mL, closed shapes) treatment. *=p<0.05 by unpaired Student's t-test or unpaired one-way ANOVA between Let-7^{WT} and Let-7^{LR} in mock and BDNF conditions; and (D) Soma size (left panel) and total dendritic length (right panel) did not significantly differ between Let-7^{WT} and Let-7^{LR} in mock or BDNF treatment. Error bars represent SEM. All experiments done in the presence of Actinomycin D. See also FIG. 13;

FIG. 7 is a proposed model for the determination of mRNA target specificity in BDNF-mediated translation: Left panel: In the absence of BDNF stimulation, both Lin28-targeted precursor miRNAs (GGAG, right side) and non-Lin28-targeted precursor miRNAs (left side) are processed into mature miRNAs. mRNAs targeted for translational repression or degradation by these mature miRNAs accumulate in P-bodies. Right panel: BDNF induces both positive and negative regulation of miRNA biogenesis. In the presence of BDNF, TRBP phosphorylation and Dicer protein levels increase leading to a general enhancement of processing of precursor miRNAs into mature miRNAs. Increased abundance of mature miRNAs leads to an increase in targeting of mRNAs for repression and increases the number of P-bodies in cells. However, Lin28a protein levels also increase in response to BDNF (far right). Because Lin28a selectively prevents processing of its targeted precursor miRNAs (GGAG) into mature miRNAs, this population of miRNAs is diminished and mRNA targets of these miRNAs are no longer efficiently repressed and become more readily available for translation. The differential effects of BDNF on distinct miRNA populations can explain the selective increase in translation of only specific mRNAs in response to BDNF;

FIGS. 8A-8I, in relation to FIG. 1, show the composition of neuronal P-bodies: (A) Endogenous Dcp1a (top panel) colocalizes with GFP-Dcp1a (middle panel) in a confocal projection of hippocampal pyramidal neuron dendrites. Bottom panel, overlaid image; (B) mCherry-tagged Dcp1a (top panel) colocalizes with GFP-tagged Ago2 (middle panel) in a confocal projection. Bottom panel, overlaid image; (C) Endogenous Rck/p54 (top panel) colocalizes with endogenous GW182 (middle panel) in a confocal projection. Bottom panel, overlaid image; (D) Consistent with the lack of translation in P-bodies, endogenous ribosomal RNA, stained with Y10b (top panel) does not colocalize with GFP-tagged Dcp1a (middle panel) in dendrites of hippocampal pyramidal neurons. Bottom panel, overlaid image. Images shown are a single confocal slice to better appreciate co-localization given the relatively larger volume of Y10b staining Inset: enlarged view of imaged region in dashed box, showing Y10b puncta closely opposed to but not colocalizing with GFP-Dcp1a puncta; (E) mCherry-tagged Dcp1a (top panel) does not colocalize with GFP-tagged Staufen (middle panel) in confocal projections from dendrites of hippocampal pyramidal neurons. Bottom panel, overlaid image. Inset: enlarged view of imaged region in dashed box, showing GFP-Staufen surrounding but not colocalizing with a GFP-Dcp1a puncta; (F) EBFP2-tagged Dcp1a (BFP-Dcp1a; top panel) colocalizes with YFP-tagged Pat1b (YFP-Pat1b; middle panel). Scale bar represents 10 μm in all images. Images are confocal z-stack projections unless otherwise indicated; and (G) Immunostaining of endogenous P-bodies with anti-GW182 antibody shows a high variability in basal dendritic P-body number that is collectively and on average shifted to greater numbers of P-bodies in BDNF-stimulated neurons. Scatter plot of endogenous P-body numbers in individual neuronal dendrites from distinct pyramidal neurons quantified from GW182 immunostaining 75 min after mock- (open circles) or BDNF-stimulation (filled circle). Bar represents mean. Mean values±SEM are 11.36±1.21 (mock), 15.48±1.26 (BDNF), p<0.05 by Student's t-test; (H) Left: Immunoblot showing GFP-Dcp1a and endogenous Dcp1a and GW182 protein levels following 45 minutes mock (−) or BDNF (+) stimulation. Right: Relative quantification of protein levels normalized to β-tubulin III; (I) BDNF rapidly enhances the colocalization of P-body components by increasing their recruitment to P-bodies, rather than by altering their synthesis. Top panel: The fraction of total fluorescence of two co-expressed P-body markers, YFP-Pat1b and BFP-Dcp1a, that co-localized was quantified from hippocampal dendrites imaged before and after BDNF stimulation using the spots function and co-localize spots tool in Imaris software (Bitplane). The fraction of colocalized fluorescence within a dendritic segment was calculated for each P-body component by first summing the aggregate fluorescence values that co-localized with the other P-body marker, then dividing this quantity by the value of the total fluorescence intensity within the dendrite for that channel, and multiplying by 100. The percent of the total fluorescence that colocalized was significantly increased for both YFP-Pat1b and BFP-Dcp1a by BDNF (hatched bars), but not mock (checkered bars) stimulation. Middle panel: The degree of colocalization within a given P-body is not changed by BDNF stimulation. While the total amount of fluorescence found in P-bodies was increased by BDNF for both YFP-Pat1b and BFP-Dcp1a, P-bodies always demonstrated a high degree of co-localization for the two P-body markers that was not significantly altered by BDNF. Percent colocalization within P-bodies was calculated by measuring the amount of fluorescence of YFP-Pat1b or BFP-Dcp1a that co-localized within defined P-body punctae, dividing this by the aggregate fluorescence value within all defined P-body punctae, and multiplying by 100. Bottom panel: Total fluorescence of YFP-Pat1b or BFP-Dcp1a within dendritic segments did not significantly change during mock (checkered bars) or BDNF (hatched bars) stimulation. Error bars represent SEM. *=p<0.05 by unpaired Student's t-test;

FIGS. 9A-9H, in relation to FIG. 2, show that loss of P-bodies in neurons lacking either GW182 or LSm5 leaves other cellular responses to BDNF intact. GW182 was chosen as an initial target for P-body disruption since it may perform a scaffolding role in P-bodies and does not possess a known enzymatic activity that could be required for general cellular function: (A) Cultured hippocampal neurons (DIV 14) were infected with replication-incompetent lentivirus expressing mCherry and shRNA targeting GW182, or a control non-target shRNA and mCherry. Immunohistochemistry for endogenous GW182 indicated effective loss of P-bodies in cells expressing GW182 shRNA (GW182KD), but not in control shRNA infected cells; (B) Immunoblot demonstrating effective knockdown of GW182 using a rabbit polyclonal antibody raised against GW182 (16 amino acid peptide of TNRC6a absent from other TNRC6 isoforms, Abcam ab84403) with no cross-reaction to isoforms TNRC6B and TNRC6C, or Ge-1, another P-body component. Neurons expressing either shRNA against GW182 (GW182KD) or scrambled shRNA (sh-control-1) received mock or BDNF stimulation in the presence of Actinomycin-D as previously described (FIG. 2). Quantification relative to the control mock condition (set as 100%) of protein levels normalized to β-tubulin is indicated under GW182 bands; (C) Immunohistochemistry for endogenous Dcp1a showed loss of P-bodies in neurons expressing GFP-DNGW182; (D) BDNF-induced activation of the cAMP response element-binding protein (CREB) transcription factor remains intact in neurons expressing GFPDNGW182. A luciferase reporter harboring cAMP response-elements (CRE) to monitor CREB activation was expressed in neurons with or without DNGW182. Mock (open bars) and BDNF (hatched bars, 100 ng/mL 3.5 hours) stimulation were performed in the absence of a transcription blocker. Luciferase activity was normalized to coexpressed constitutive β-galactosidase activity and plotted relative to the mock condition; (E) mRNA abundance in neurons as measured by RT-qPCR with all conditions normalized to mock stimulation (open bars; set as 1.0). mRNA levels of BDNF-upregulated targets were unaltered from basal values (mock) by BDNF stimulation (BDNF; 100 ng/mL, 2 hours), or by GW182 knockdown (GW182KD, left panel) or GFP-DNGW182 expression (right panel). The BDNF-induced downregulation of KCC2 transcripts was abolished by loss of GW182 function (right panel). All RT-qPCR reactions were normalized to (3-tubulin III (neuron-specific isoform) values, which are unchanged by BDNF, within individual cDNA samples to control for consistency between amplification assays. Expression of control shRNA (sh-control-1, left panel) or GFP (right panel) serve as controls for GW182KD or GFP-DNGW182, respectively. All experiments done in the presence of Actinomycin-D to isolate changes due only to translation; (F) Immunohistochemical staining for endogenous P-body component LSm5 (middle) showed extensive colocalization with another endogenous P-body component, GW182 (top panel; overlay, bottom panel); (G) Immunohistochemistry for endogenous Dcp1a showed loss of P-bodies in neurons expressing shRNA against LSm5 (LSm5 KD), but not in control-shRNA-expressing neurons (sh-Control-2). GFP expression served to visualize neuron morphology. Scale bars represent 10 μm in all images; and (H) Increased total protein synthesis in response to BDNF is unaltered after P-body disruption by loss of LSm5. Total protein synthesis was assayed by measuring ³⁵S incorporation in sh-Control-2 and LSm5 KD neurons, undergoing mock (− BDNF) or BDNF (+ BDNF) stimulation 100 ng/mL for 2 hours, in the presence of Actinomycin-D (0.5 μg/mL, pre-incubated 10 min before stimulation). *=p<0.05 by unpaired Student's t-test;

FIGS. 10A and 10B, in relation to FIG. 2, shows that the transcription inhibitor Actinomycin-D does not alter BDNF-induced protein synthesis under the assayed conditions: (A) Immunoblotting for BDNF targets in cultured neurons treated mock (−) or BDNF (+; 100 ng/mL for 2 hours) stimulation in the absence (− Act-D) or presence of Actinomycin-D (+ Act-D; 0.5 μg/mL added 10 min before stimulation). Arc demonstrates strong transcription-dependent upregulation by BDNF and serves as an indicator for Actinomycin-D efficacy; and (B) Protein levels of BDNF targets relative to the mock-stimulated condition (open bars, set as 1.0). Quantification is by densitometry with internal normalization to β-tubulin. Error bars represent SEM. *=p<0.05 by unpaired Student's t-test;

FIGS. 11A-11B, in relation to FIG. 2 and FIG. 3, are control experiments supporting involvement of the miRNA pathway in BDNF-regulation of protein synthesis: (A) Activity of a luciferase reporter lacking binding sites is not affected by expression of shRNA targeting CXCR4. Luciferase reporter assay was carried out (as in FIG. 3) in neurons expressing a control luciferase reporter free of any binding site for shRNA against CXCR4 (sh-CXCR4) and treated with mock (−BDNF) or BDNF (+ BDNF) stimulation at 100 ng/mL for 2 hours in the presence of actinomycin-D (0.5 μg/mL, added 20 min before stimulation); and (B) Global activation of miRNA biogenesis by Dicer stabilization with enoxacin does not significantly alter the quantity of total cellular protein synthesis. Total protein synthesis was assayed by measuring ³⁵S incorporation in neurons, undergoing mock or BDNF stimulation (100 ng/mL for 2 hours), with or without enoxacin, in the presence of actinomycin-D (0.5 μg/mL, added 20 min before stimulation). Oxalinic acid, a structurally similar control for enoxacin, also does not alter total cellular protein synthesis. Error bars represent SEM. *=p<0.05 by unpaired Student's t-test;

FIG. 12, in relation to FIG. 4, shows binding sites for Lin28-regulated miRNAs (let-7, miR-143, and miR-107) in the 3′UTRs of example BDNF-upregulated targets, CaMKIIα and GluR1. The binding region for the miRNA seed sequence (nucleotides 2-7 of the miRNA) is underlined. Complementary base pairing is denoted by uppercase letters, noncomplementary base pairing are in lowercase letters, and G:U wobbles are in gray font. Base pair position indicates the base in the 3′UTR of the mRNA where the miRNA 5′ end binds. microRNA seeds having an average phylogenetic conservation score higher than 0.8 are denoted by the pound (#) sign. The conservation score is based on alignment of the longest 3′UTR for 17 vertebrates, including mammalian, amphibian, bird and fish species. Conservation scores range from 0 to 1, with 1 being perfectly conserved and scores over 0.8 predictive of functional relevance (PITA) (Kertesz et al., 2007);

FIGS. 13A-13C, in relation to FIG. 6, shows that loss of P bodies in neurons expressing dominant-negative form of GW182 (DNGW182) inhibits BDNF-induced dendritic arborization: (A) Representative images of neurons expressing untagged mCherry and either GFP (top panels) or GFP-DNGW182 (bottom panels) following 72 hrs of mock- or BDNF- (25 ng/mL) treatment. Images show channels (mCherry) only. Scale bar represents 50 μm; (B) Sholl analysis representing dendritic complexity at increasing distances from the cell body for GFP- (circles) or GFP-DNGW182- (triangles) expressing neurons following mock- (open shapes) or BDNF-(25 ng/mL, closed shapes) treatment. *=p<0.05 by unpaired one-way ANOVA; Mann-Whitney U test modified Bonferroni correction; and (C) Total dendritic length (top panel) and soma size (bottom panel) are not significantly different between control and GFP-DNGW182-expressing neurons following mock or 25 ng/mL BDNF treatment. Error bars represent SEM. mock, n=30 cells; BDNF, n=27; GFP-DNGW182, mock, n=21; GFPDNGW182, BDNF, n=27;

FIG. 14 shows a representative diagram of a mutation introduced into a Let-7 microRNA; and

FIG. 15 is a diagram of a representative viral expression construct for the presently disclosed mutated Lin28-resistant pre-Let-7 microRNA.

### DETAILED DESCRIPTION

The presently disclosed subject matter now will be described more fully hereinafter with reference to the accompanying Figures, in which some, but not all embodiments of the presently disclosed subject matter are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

I. Expression Constructs and Methods for Making a Developmental Timing Regulator Protein-Resistant PRECURSOR miRNA

Control of translation is a fundamental source of regulation in gene expression. The induction of protein synthesis by brain-derived neurotrophic factor (BDNF) critically contributes to enduring modifications of synaptic function, but how BDNF selectively affects only a minority of expressed mRNAs is poorly understood.

The presently disclosed subject matter provides, in some embodiments, that BDNF rapidly elevates Dicer, increasing mature miRNA levels and inducing RNA-processing bodies in neurons. BDNF also rapidly induces Lin28, causing selective loss of Lin28-regulated miRNAs and a corresponding upregulation in translation of their target mRNAs. Binding sites for Lin28-regulated miRNAs are necessary and sufficient to confer BDNF-responsiveness to a transcript. Lin28 deficiency, or expression of a Lin28-resistant Let-7 precursor miRNA (DNA sequence shown as SEQ ID NO: 10), inhibits BDNF translation specificity and BDNF-dependent dendrite arborization.

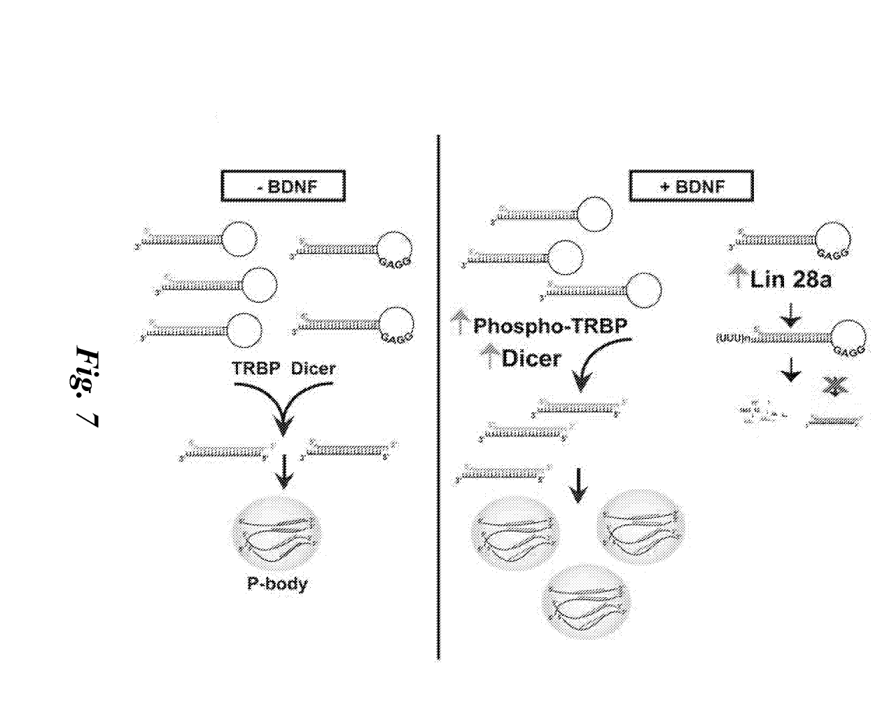

The capacity to rapidly alter the abundance of effector proteins through regulating translation is critical to the biological actions of multiple stimuli. The pathways that mediate stimulus-dependent selection of specific mRNAs for enhanced translation, however, have remained poorly understood. The presently disclosed subject matter provides a novel coordinated mechanism for genome-wide control of translation specificity that involves stimulus-dependent positive and negative regulation of miRNA biogenesis (model, FIG. 7). Direct evidence is provided that BDNF achieves translation target specificity by elevating levels of both Dicer and Lin28a proteins in a rapid and transcription-independent manner. The resultant action of Dicer and Lin28a on the cellular profile of miRNAs in response to BDNF effectively determines which mRNAs will participate in translation or be excluded through GW182-associated repression.

The presently disclosed results provide the following insights into specificity for BDNF up- and down-regulated protein synthesis: First, upregulation of an mRNA's translation by BDNF requires the target mRNA to be repressed and enriched in association with P-body component GW182 under basal conditions. Interference with Dicer or GW182 function prevents this basal repression and therefore occludes stimulus-dependent induction of translation. Second, interference with Dicer or GW182 blocks the downregulation of target mRNA translation by BDNF. Third, the presence of seed-matched sites for a Lin28-regulated miRNA within the 3′UTR are predictive of an upregulated BDNF-target mRNA. Interference with selective Lin28-mediated pre-miRNA degradation blocks the induction of targets upregulated by BDNF. Fourth, the stimulus-induced association of an mRNA with GW182 is reciprocally related to its level of translation. BDNF diminishes the GW182 association of mRNA for translationally upregulated targets and enhances the GW182 association for downregulated targets.

The collective evidence disclosed herein indicates that GW182 interaction with miRNAs and RISC components can trigger the formation of P-bodies as sites where repressed mRNAs accumulate (Eulalio et al., 2007; Liu et al., 2005b). Without wishing to be bound to any one particular theory, it is thought that the miRNA-dependent repression induced by BDNF typically employs P-bodies, consistent with the striking Dicer-dependent increase in P-body number in response to BDNF. Loss of visible P-bodies by LSm5 knockdown, however, produced no apparent interference with the specificity of BDNF-induced protein synthesis, and the presently disclosed data also could be consistent with a model in which the mRNA repression does not occur in P-bodies per se, but elsewhere in a GW182- and miRNA-dependent manner

BDNF-induced repression of mRNAs involves the rapid Dicer-dependent appearance of P-bodies in neuronal cell soma and dendrites that can occur independently of new transcription and, as reported in other cell types (Teixeira et al., 2005) appears to result from coalescence of existing P-body components. Consistent with these results, both Dicer and pre-miRNAs are present in dendrites and isolated synapses (Lugli et al., 2008), suggesting that trafficking might not be required for rapid responses; whether BDNF regulates neuronal miRNA biogenesis on a subcellular level remains to be investigated.

Recent work indicates that many miRNAs can turnover more quickly in neurons than in other cell types. miRNAs from brain or from hippocampal cultures have variable estimated half-lives of 0.5-6 hr (Krol et al., 2010; Sethi and Lukiw, 2009), compared with half-lives≧24 hr in non-neuronal cells. This property might allow degradation of a pre-miRNA species in neurons to rapidly lower the corresponding mature miRNA level, as supported by the presently disclosed finding of rapid Lin28-mediated decline in mature Let-7 miRNAs. When miRNA precursors are not depleted by BDNF-induced Lin28, the available precursors (i.e., pri- and pre-miRNAs) appear sufficient to replenish mature Let-7 levels even when transcription is blocked for 1 to 2 hr.

miRNAs have been reported to repress target mRNA by inhibition of translation or by degradation. miRNA-dependent degradation of target mRNA was observed for representative mRNA targets that underwent decreased translation in response to BDNF; these findings are consistent with studies citing mRNA destabilization as a predominant source of miRNA-dependent reductions in protein (Guo et al., 2010; Hendrickson et al., 2009). However, the presently disclosed data also suggest that miRNAs can function by translation suppression in neurons under basal conditions. Specifically, BDNF-upregulated targets were repressed and associated with GW182 prior to BDNF stimulation. Disruption of this basal repression (by deficiency of GW182 or Dicer) increased protein production from BDNF-upregulated targets with no detectable elevation of their mRNA levels, consistent with reports of miRNA function by inhibition of translation (Chendrimada et al., 2007; Mathonnet et al., 2007; Petersen et al., 2006). In addition to its established role in tuning protein levels, the presently disclosed data highlight a role for miRNA-mediated repression in determining the specificity of stimulus-induced protein synthesis through both translation inhibition and mRNA degradation.

Mammalian Lin28 is reported to be downregulated during development with little or no expression in differentiated cells such as neurons (Moss and Tang, 2003). The presently disclosed data similarly indicate low basal Lin28 expression in mature neurons, but show that BDNF induces rapid transcription-independent upregulation of Lin28a, which alters levels of Lin28-targeted miRNAs and might also perform additional functions. Lin28 expression has been associated with oncogenesis and, in conjunction with other modulators, also can induce pluripotent stem cells from differentiated tissues (Viswanathan et al., 2009; Yu et al., 2007). This underscores the concept that the reprogramming of gene expression accompanying both neoplastic transformation and induced pluripotency states may, at least in part, be additionally shared by the induction of plasticity in the adult nervous system.

Collectively, the presently disclosed data indicate that miRNA biogenesis undergoes dynamic post-transcriptional regulation in neurons to impart mRNA selection for BDNF-dependent protein synthesis. The presently disclosed findings also reveal a role for mRNA repression in association with the P-body component GW182 in conferring specificity to basal, as well as stimulus-dependent translation through miRNA-dependent regulation. It is likely that other stimuli use distinct or overlapping regulatory mechanisms in the miRNA biogenesis pathway to generate specificity in the post-transcriptional regulation of gene expression.

Accordingly, stimulus-dependent change in the complement of proteins expressed by neurons underlies enduring alterations in synaptic response and ultimately shapes brain function. The neurotrophin, BDNF, enhances protein synthesis, but demonstrates a remarkable degree of transcript selectivity. The presently disclosed data establish a mechanism for this specificity through the two-part post-transcriptional control of miRNA biogenesis. BDNF-induction of Dicer results in the miRNA-mediated repression of many mRNAs, while concomitant induction of Lin28 by BDNF downregulates specific miRNAs to allow the increased translation of select mRNAs.

As such, the presently disclosed subject matter provides precursor microRNA molecules that have been modified or mutated to prevent a protein involved in regulating developmental timing, oncogenesis, and/or neuronal growth from blocking processing of the precursor microRNA sequence to the mature microRNA. In other words, the mutation results in a precursor microRNA molecule that cannot be blocked by a protein involved in regulating developmental timing, oncogenesis, and/or neuronal growth from processing to the mature microRNA.

As used herein, the terms “microRNA,” “miRNA,” or “miR” are synonymous and are used interchangeably and include human miRNA, mature single stranded miRNA, precursor miRNA (pre-miRNA), and variants thereof. In some instances, the terms also include primary miR transcripts and duplex miRNA. The sequences for particular miRNA, including human mature and precursor sequences, can be found in several publicly available database including, but not limited to, the miRBase database (accessible at http://www.mirbase.org). For certain miRNA, a single precursor contains more than one mature miRNA sequence. In other instances, multiple precursor miRNA contain the same mature sequence. In some instances, mature miRNA have been renamed based on new scientific consensus. One of ordinary skill in the art appreciates that scientific consensus regarding the precise nucleic acid sequence for a given miRNA, in particular for mature forms of the miRNA, may change with time.

Accordingly, in some embodiments, the presently disclosed subject matter provides a precursor microRNA molecule, the precursor microRNA molecule comprising a nucleic acid comprising: (a) a precursor microRNA sequence; and (b) a mutation in the sequence that prevents a protein involved in regulating developmental timing, oncogenesis, and/or neuronal growth from blocking processing of the precursor microRNA sequence to a mature microRNA.

The term “nucleic acid” or “polynucleotide” refers to a polymeric form of nucleotides of any length, either ribonucleotides and/or deoxyribonucleotides. These terms include a single-, double- or triple-stranded DNA, genomic DNA, cDNA, RNA, DNA-RNA hybrid, or a polymer comprising purine and pyrimidine bases, or other natural, chemically, biochemically modified, non-natural or derivatized nucleotide bases. The backbone of the nucleic acid can comprise sugars and phosphate groups (as may typically be found in RNA or DNA), or modified or substituted sugar or phosphate groups. Alternatively, the backbone of the nucleic acid can comprise a polymer of synthetic subunits such as phosphoramidates and thus can be an oligodeoxynucleoside phosphoramidate (P—NH₂) or a mixed phosphoramidate-phosphodiester oligomer. In addition, a double-stranded nucleic acid can be obtained from the single stranded nucleic acid product of chemical synthesis either by synthesizing the complementary strand and annealing the strands under appropriate conditions, or by synthesizing the complementary strand de novo using a DNA polymerase with an appropriate primer.

The following are non-limiting examples of nucleic acids: a gene or gene fragment, exons, introns, mRNA, tRNA, rRNA, ribozymes, cDNA, recombinant nucleic acids, branched nucleic acids, plasmids, vectors, isolated DNA of any sequence, isolated RNA of any sequence, nucleic acid probes, and primers. A nucleic acid may comprise modified nucleotides, such as methylated nucleotides and nucleotide analogs, uracyl, other sugars and linking groups such as fluororibose and thioate, and nucleotide branches. The sequence of nucleotides may be interrupted by non-nucleotide components. A nucleic acid may be further modified after polymerization, such as by conjugation with a labeling component. Other types of modifications included in this definition are caps, substitution of one or more of the naturally occurring nucleotides with an analog, and introduction of means for attaching the nucleic acid to proteins, metal ions, labeling components, other nucleic acids, or a solid support.

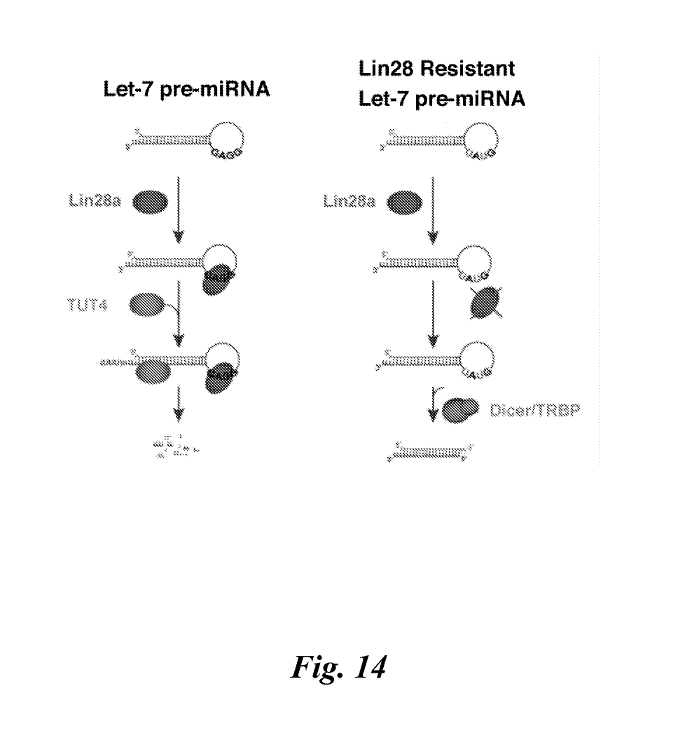

In one embodiment, the precursor microRNA sequence of the presently disclosed subject matter is Let-7 (SEQ ID NO: 10). As an example, DNA encoding the Homo sapiens microRNA let-7a-1 sequence (NCBI Reference Sequence: NR_{—}029476; SEQ ID NO: 9) can be used as a template to mutate the sequence from the nucleotide sequence GGAG (nucleotides 49-52 in SEQ ID NO:9) to the nucleotide sequence GTAT (nucleotides 49-52 in SEQ ID NO:10) resulting in a precursor microRNA with the nucleotide sequence change GUAU (FIG. 14; SEQ ID NO: 12). Therefore, in one embodiment, the mutation is a change from the nucleotide sequence GGAG to the nucleotide sequence GUAU. In a further embodiment, the mutation is in the part of the precursor microRNA sequence that forms a terminal loop structure.

In the presently disclosed subject matter, the protein involved in regulating developmental timing, oncogenesis, and/or neuronal growth is any protein that recognizes the conserved “GGAG” motif in the terminal loop of a precursor microRNA. In a particular embodiment, the protein involved in regulating developmental timing, oncogenesis, and/or neuronal growth is Lin28.

Lin28 consists of Lin28a and Lin28b in mammals. These are the RNA-binding proteins that bind to the ‘GGAG’ sequence, which is mutated by the presently disclosed methods, thereby preventing their binding. Lin28 proteins are not only involved in developmental timing, but also promote growth in other contexts. For example, they are reported to be overexpressed in approximately 15% of cancers, and are expressed in the neurons to promote growth. Thus, Let-7LR could be useful in multiple biological contexts, including therapeutic methods, such as treatments for various cancers, including brain cancers.

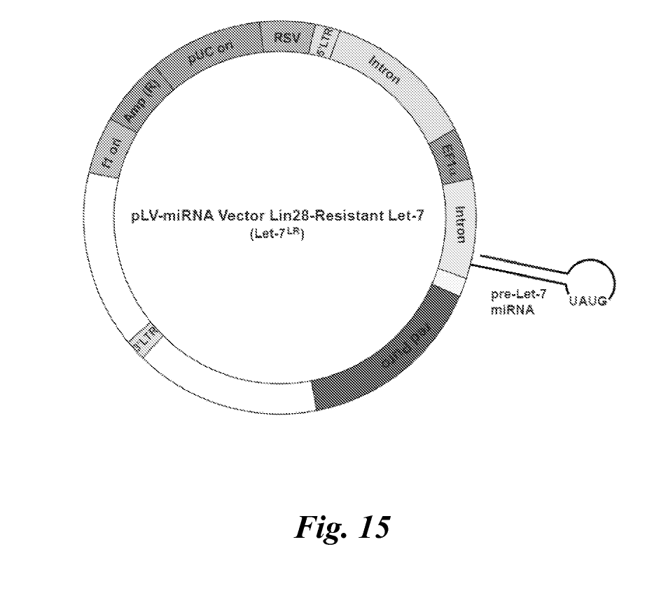

The presently disclosed subject matter also provides a DNA molecule encoding a precursor microRNA molecule. In an embodiment, the DNA molecule encodes Let-7 (SEQ ID NO: 10). In another embodiment, the DNA molecule is operably linked to a recombinant expression vector. In a further embodiment, the vector is a lentiviral vector. A diagram of a representative viral expression construct for the presently disclosed mutated Lin28-resistant pre-Let-7 microRNA is provided in FIG. 15. Accordingly, in some embodiments, the presently disclosed precursor microRNA sequence can be inserted into a viral backbone, thereby allowing production of virus encoding the precurosor miRNA and an efficient means (e.g., by viral infection) to introduce the precurosor miRNA for cellular expression.

As used herein, the term “operably linked” means that nucleic acid sequences or proteins are operably linked when placed into a functional relationship with another nucleic acid sequence or protein. For example, a promoter sequence is operably linked to a coding sequence if the promoter promotes transcription of the coding sequence. As a further example, a repressor protein and a nucleic acid sequence are operably linked if the repressor protein binds to the nucleic acid sequence. Additionally, a protein may be operably linked to a first and a second nucleic acid sequence if the protein binds to the first nucleic acid sequence and so influences transcription of the second, separate nucleic acid sequence.

Generally, “operably linked” means that the DNA sequences being linked are contiguous, although they need not be, and that a gene and a regulatory sequence or sequences (e.g., a promoter) are connected in such a way as to permit gene expression when the appropriate molecules (e.g., transcriptional activator proteins˜transcription factors˜or proteins which include transcriptional activator domains) are bound to the regulatory sequence or sequences.

As used herein, the term “vector” refers to a nucleic acid construct designed for transduction/transfection of one or more cell types. Vectors may be, for example, “cloning vectors,” which are designed for isolation, propagation and replication of inserted nucleotides, “expression vectors,” which are designed for expression of a nucleotide sequence in a host cell, a “viral vector,” which is designed to result in the production of a recombinant virus or virus-like particle, or “shuttle vectors,” which comprise the attributes of more than one type of vector. The term “replication” means duplication of a vector.

The term “expression vector” is used interchangeably herein with the term “plasmid” and “vector” and refers to a recombinant DNA molecule containing a desired coding sequence and appropriate nucleic acid sequences necessary for expression of the operably linked coding sequence (e.g., an insert sequence that codes for a product) in a particular host cell. The term “plasmid” refers to an extrachromosomal circular DNA capable of autonomous replication in a given cell. In certain embodiments, the plasmid is designed for amplification and expression in bacteria. Plasmids can be engineered by standard molecular biology techniques. See Sambrook et al., Laboratory Manual, Cold Spring Harbor Laboratory Press, Cold Spring Harbor (1989), N.Y. Nucleic acid sequences necessary for expression in prokaryotes usually include a promoter, an operator (optional), and a ribosome binding site, often along with other sequences.

Further, in other embodiments, the presently disclosed subject matter provides a cell comprising one or more of: (a) a precursor microRNA molecule, the precursor microRNA molecule comprising a nucleic acid comprising: (i) a precursor microRNA sequence; and (ii) a mutation in the sequence that prevents a protein involved in regulating developmental timing, oncogenesis, and/or neuronal growth from blocking processing of the precursor microRNA sequence to a mature microRNA; and (b) a DNA molecule encoding the presently disclosed precursor microRNA molecule. In an embodiment, the cell is a mammalian cell. In another embodiment, the cell is a human cell.

Also provided by the presently disclosed subject matter is a kit comprising a DNA molecule encoding a precursor microRNA molecule.

The presently disclosed subject matter further provides methods for making a precursor microRNA molecule that has been modified or mutated to prevent a protein involved in regulating developmental timing, oncogenesis, and/or neuronal growth from blocking processing of the precursor microRNA sequence to the mature microRNA.

Accordingly, in one embodiment, a method is provided for making a precursor microRNA molecule such that the ability of a protein involved in regulating developmental timing, oncogenesis, and/or neuronal growth to decrease the level of the precursor microRNA molecule is blocked, the method comprising: (a) providing a precursor microRNA sequence; (b) making a mutation in the precursor microRNA sequence, wherein the ability of a protein involved in regulating developmental timing, oncogenesis, and/or neuronal growth to decrease the level of the microRNA molecule is blocked. In an embodiment, the precursor microRNA sequence is Let-7 (SEQ ID NO: 9). In another embodiment, the protein involved in regulating developmental timing, oncogenesis, and/or neuronal growth is Lin28. In still another embodiment, the mutation is in the part of the precursor microRNA sequence that forms a terminal loop structure. In a further embodiment, the mutation is a change from the nucleotide sequence GGAG to the nucleotide sequence GUAU. In a still further embodiment, the mutation is made by performing PCR site-directed mutagenesis.

In an embodiment, the method further comprises expressing the mutated precursor microRNA sequence in a cell. In another embodiment, the cell is a mammalian cell. In still another embodiment, the cell is a human cell.

II. Methods for Using the Presently Disclosed Precursor miRNA Molecules

The presently disclosed subject matter provides methods for using the presently disclosed precursor microRNA constructs. Expression of these constructs can be used for many purposes including, but not limited to, blocking the ability of a protein involved in regulating developmental timing, oncogenesis, and/or neuronal growth to decrease the levels of precursor microRNA, to act as a dominant negative for the effects of a developmental timing regulator protein on mature microRNA levels, to implicate developmental timing regulator protein regulation of microRNA levels in a biological process, and to elevate cellular levels of a microRNA.

In an embodiment, the presently disclosed subject matter provides a method for elevating a level of a microRNA in a cell by making a precursor microRNA molecule, the method comprising: (a) providing a precursor microRNA sequence; (b) making a mutation in the precursor microRNA sequence such that the ability of a protein involved in regulating developmental timing, oncogenesis, and/or neuronal growth to decrease the levels of the microRNA molecule is blocked; and (c) expressing the precursor microRNA molecule in a cell. In an embodiment, the precursor microRNA sequence is Let-7 (SEQ ID NO: 12). In another embodiment, the protein involved in regulating developmental timing, oncogenesis, and/or neuronal growth is Lin28. In a further embodiment, the mutation is in the part of the precursor microRNA sequence that forms a terminal loop structure. In still another embodiment, the mutation is a change from the nucleotides GGAG to the nucleotides GUAU. In an embodiment, the mutation is made by performing PCR site-directed mutagenesis.

In representative embodiment, the method further comprises expressing the mutated precursor microRNA sequence in a cell. The cell according to the methods of the presently disclosed subject matter may be a mammalian cell, such as a human cell. In an embodiment, the method of expressing the precursor microRNA sequence in a cell occurs with a vector comprising a DNA molecule encoding the precursor microRNA. In another embodiment, the vector is a lentiviral vector. In an embodiment, the methods for elevating levels of a microRNA in a cell results in inhibition of brain-derived neurotrophic factor (BDNF) translation specificity. In another embodiment, brain-derived neurotrophic factor (BDNF)-dependent dendrite arborization is inhibited.

In further embodiments, the presently disclosed subject matter provides a method for treating a cancer, the method comprising administering to a subject in need of treatment thereof, a therapeutically effective amount of a precursor microRNA comprising a mutation in the precursor microRNA sequence that prevents a protein involved in regulating developmental timing, oncogenesis, and/or neuronal growth from blocking processing of the precursor microRNA sequence to a mature microRNA. In some embodiments, the cancer is a brain cancer.

In some embodiments, the precursor microRNA is Let-7 (SEQ. ID. NO. 12). In some embodiments, the a protein involved in regulating developmental timing, oncogenesis, and/or neuronal growth in Lin28.

The term “effective amount,” as in “a therapeutically effective amount,” of a therapeutic agent refers to the amount of the agent necessary to elicit the desired biological response. As will be appreciated by those of ordinary skill in this art, the effective amount of an agent may vary depending on such factors as the desired biological endpoint, the agent to be delivered, the composition of the pharmaceutical composition, the target tissue or cell, and the like. More particularly, the term “effective amount” refers to an amount sufficient to produce the desired effect, e.g., to reduce or ameliorate the severity, duration, progression, or onset of a disease, disorder, or condition, or one or more symptoms thereof; prevent the advancement of a disease, disorder, or condition, cause the regression of a disease, disorder, or condition; prevent the recurrence, development, onset or progression of a symptom associated with a disease, disorder, or condition, or enhance or improve the prophylactic or therapeutic effect(s) of another therapy.

The subject treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing disease, disorder, condition or the prophylactic treatment for preventing the onset of a disease, disorder, or condition or an animal subject for medical, veterinary purposes, or developmental purposes. Suitable animal subjects include mammals including, but not limited to, primates, e.g., humans, monkeys, apes, gibbons, chimpanzees, orangutans, macaques and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, guinea pigs, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a disease, disorder, or condition. Thus, the terms “subject” and “patient” are used interchangeably herein. Subjects also include animal disease models (e.g., rats or mice used in experiments, and the like).

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, parameters, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### Example 1

### Experimental Procedures

### Hippocampal Cultures and Stimulation:

Dissociated hippocampal cultures were prepared from postnatal day 0 (P0) mice as previously described (Meffert et al., 2003). Knockdown was by lentiviral-mediated delivery of shRNA to cultures at multiplicity of infection of 5-10, 48 hr before imaging or 4-5 days for GW182. Cultures were preincubated 10-20 min and mock- or BDNF-stimulated (100 ng/mL BDNF) in the presence of Actinomycin-D (0.5 μg/mL), unless indicated otherwise.

### Imaging and Quantification

Confocal images of hippocampal pyramidal neurons were acquired on either a Yokogawa spinning disk (Zeiss) at 37° C. (live cells), or a LSM5 Pascal system (fixed cells). Laser power and exposure time were adjusted to minimize photobleaching and avoid saturation. All experiments were from a minimum of 3 independent cultures and no more than 3 neurons per dish.

Dissociated hippocampal cultures were prepared from postnatal day 0 (P0) mice as previously described (Meffert et al., 2003) and were maintained in Neurobasal A medium (Gibco, 10888) with B27 Supplement (Gibco 17504-44). Neurons were transiently transfected with Lipofectamine 2000 according to the manufacturer's instructions (Invitrogen) 24 hours before experimentation. For live cell imaging, confocal images of hippocampal pyramidal neurons (excitatory, determined by morphology) in 0.24-0.3 μm Z sections were acquired using a 40×, 1.3 NA, EC Plan Neofluoar (whole cells) or a 100×, 1.4 NA Plan Apochromat oil immersion objective (dendrites) on a Yokogawa spinning disk system (Cell Observer, Carl Zeiss) at 37° C. in Tyrodes buffer (in mM: 119 NaCl, 5 KCl, 2 CaCl₂, 0.2 MgCl₂, Glucose, 25 Hepes, 0.01 Glycine, pH 7.4). EBFP2 was excited at 405 nm and emissions collected at 425-475 nm. GFP was excited at 488 nm and emissions collected at 500-550 nm; mCherry was excited at 561 nm and emissions collected at 598-660 nm. Laser power and exposure time were adjusted to minimize phototoxicity and avoid saturation. All experiments were from a minimum of 3 independent cultures, and no more than 3 neurons per dish; the experimenter was blinded to conditions during analysis.

For fixed cells, confocal images were acquired in 0.3 μm (dendrites) or 1.7 μm (whole cells for arborization) Z sections on an LSM5 Pascal system (Carl Zeiss) using a 63×, 1.4 NA Plan Apochromat oil immersion objective and 2× optical zoom (dendrites) or using a 25×, 0.80 NA Plan-Neofluor multi-immersion objective and 0.7×-1× optical zoom (for whole cells). GFP was excited at 488 nm and emissions collected at 505-530 nm; mCherry was excited at 543 nm and emissions collected above 560 nm. Laser power and exposure time were adjusted to minimize photobleaching and avoid saturation. All experiments were from a minimum of 3 independent cultures and no more than 3 neurons per dish Z-stacks containing the entire neuron or process of interest were analyzed using Imaris 7.0.0 (Bitplane) and ImageJ software. Automated analysis of P-body numbers was conducted using Spots detection in Imaris. A quality filter and intensity median filter for the channel were used to restrict detection of puncta within dendrites only. Colocalization analysis was performed using the Colocalize Spots function. The percent colocalization of P-body components was calculated by subtracting the number of colocalized BFP-Dcp1a or YFP-Pat1b puncta from the total number of BFP-Dcp1a or YFP-Pat1b puncta and multiplied by 100. The percent of colocalized fluorescence was calculated for each P-body component by first summing the aggregate fluorescence values that co-localized with the other P-body marker in ‘spots’, then dividing this quantity by the value of the total fluorescence intensity within the dendrite for that channel, and multiplying by 100. The Surfaces tool in Imaris was used to create a representation of the dendrite in order to determine total fluorescence intensity corresponding to the dendrite region alone. The channel (soluble mCherry expression) was used as the source channel to compute the Surfaces. Total fluorescence of a dendritic segment was calculated by summing the intensity fluorescence values of all of the Surfaces representing a single dendrite. Sholl analysis was performed using the Sholl analysis plugin in ImageJ (A. Ghosh lab) from Z-compressed projections traced semi-automatically in NeuronJ. For analysis, dendritic intersections were counted using a circle of 15 μm diameter centered on the cell soma and subsequent circles of increasing 5 μm diameter increments.

RNA analysis

For RT-qPCR, TaqMan Gene Expression and MicroRNA Assays (Applied Biosystems) were performed with quantitation by the standard-curve method and no preamplification, RQ was calculated as 2^{-LCtBDNF}/2^{-LCtmock} where LCt=(cycle threshold for miRNA of interest)−(cycle threshold for reference control).

### Reporter Assays

The following CXCR4 siRNA/miRNA reporter assay constructs (Addgene) were used: siRNA reporter (PCD FLIP, Plasmid 12567), miRNA reporter (PCD FL4X, 12565), control luciferase reporter (PCD FLOX, 12563), and CXCR4 shRNA (pLKO.1 puro CXCR4 siRNA-2, 12272) (Wang et al., 2006). Let-7 luciferase reporters with wild-type or mutated Let-7 miRNA binding sites were gifts from G. Hannon (Liu et al., 2005b). 3′UTR reporters were constructed by inserting 3′UTRs from GluR1, CaMKIIu or KCC2 downstream of luciferase in pGL3-Control vector (Promega). A Lin28-resistant Let-7 pre-miRNA was generated by mutation of the conserved Lin28 “GGAG” recognition motif to “GtAt” in the terminal loop of pLV-hsa-let-7a-1 (Biosettia).

### Immunoblotting

Primary cultures of mouse hippocampal neurons (DIV 14˜15) were incubated in serum-reduced medium (0.5% B27 supplement) for 2 hours, followed by 0.5 mg/mL Actinomycin-D for 10-20 min. Bath application of BDNF (100 ng/mL) was for designated periods (5 min-2 hours). The cultures were washed 3 times and harvested on ice with lysis buffer (50 mM Hepes, 150 mM NaCl, 10% Glycerol, 1 mM EDTA, 1% Triton-X-100, 0.2% SDS) plus freshly added protease inhibitor cocktail (Roche) and PMSF. Protein concentration was determined by Bradford Assay. If required, lysates were treated with Lambda Protein Phosphatase according to manufacturer's instructions (New England Biolabs P0753S). Equal amounts of lysate protein were resolved on SDS-PAGE gels, and electrotransferred to PVDF membrane. Membrane was blocked with 5% milk in Tris-buffered saline tween 20 (TBST) and probed with primary antibodies in 5% milk or BSA in TBST: GluR1 (Millipore AB1504), CaMKIIα (Zymed 13-7300), Homer2 (gift of P. Worley), KCC2 (Upstate 07-432), Kv1.1 (NeuroMab 73-007), β-tubulin (U. Iowa DSHB, clone E-7), GAPDH (gift of S. Snyder), GW182 (18033 gift of M. Fritzler or Abcam ab84403), Dcp1a (gift of J. Lykke-Andersen or NeuroMab clone3G4), phospho-ERK ½ (Sigma M 7802), Dicer (NeuroMab clone N167/7), TRBP (Abeam ab72110), Lin28a (Cell Signaling A177), Lin28b (gift of E. Moss or Cell Signaling 5422), Arc (SantaCruz 17839), mCherry (Clontech 632496), GFP (NeuroMab N86/8).

### ³⁵S-Labelling

Cultured neurons were pre-incubated in media containing reduced-serum and Actinomycin-D as previously described, followed by washing and incubation for 10 min with methionine- and cysteine-free DMEM (Mediatech, Inc.), and ³⁵S labeling in the same DMEM with the addition of ³⁵5-methionine/cysteine (³⁵S Met/Cys EasyTag Mix, Perkin Elmer) to a final concentration of 100 fCi/mL. Mock- or BDNF stimulation was for 2 hours. Cells were washed and lysed with lysis buffer (see immunoblotting). Lysates were centrifuged and collected supernatants subjected to Bradford assay. To assess newly synthesized proteins, 200-500 μg of lysates proteins were precipitated with 10% trichloroacetic acid (TCA) for 1 hour on ice in the presence of 0.5% deoxycholate (DOC) to remove interfering phenol red. After centrifugation, protein pellets were washed with ice-cold 95% ethanol, solubilized in denaturing buffer (50 mN Tris pH 8.3, 5 mM EDTA, 0.05% SDS, 6M urea), and subjected to liquid scintillation counting (Econofluor, New England Nuclear, Inc.). ³⁵S Disintegration per minute (DPM) was used to quantitate protein synthesis after subtraction of background readings.

### Immunopurification of GW182

Proteins and mRNAs associated with P-body component GW182 were isolated through immunoprecipitation of GW182 by modification of previously published protocols (Keene et al., 2006; Moser et al., 2009). Primary cultures of mouse hippocampal neurons (DIV 14-15) were incubated in serum-reduced medium (0.5% B27 supplement) for 2 hours, followed by 0.5 mg/mL Actinomycin-D for 1030 min and mock or BDNF-stimulation for 30˜60 min. Cell lysates were harvested in polysomal lysis buffer (100 mM KCl, 5 mM MgCl₂, 10 mM HEPES pH 7.0, 0.5% NP-40) with protease inhibitor cocktail and freshly added 20 mM EDTA, 1 mM DTT, 100 U/mL RNase inhibitor (RNaseOut, Invitrogen) and 400 μM Vanadyl ribonucleoside complexes (SIGMA). Lysates were centrifuged and the supernatants pre-cleared by one-hour incubation with recombinant protein G beads pre-washed in NT2 buffer (50 mM Tris-HCl pH 7.4, 150 mM NaCl, 1 mM MgCl₂, 0.05% NP-40). Antibody coating of protein G beads was carried out with antiserum against GW182 (18033, gift of M. Fritzler) or control isotype-specific serum in NT2 buffer plus protease inhibitor for 4 hours after blocking with NT2 buffer plus 5% BSA and 1 mg/mL heparin for 1 hour. For immunoprecipitation (IP), equal amounts of lysate protein (2-5 mg) was incubated with antibody-coated beads and tumbled for 4 hours at 4° C., followed by washing. RNAs were recovered from GW182 immunoprecipitates by Tri-Reagent as described below.

Recovered RNAs were resuspended in nuclease-free water, measured for RNA concentration, and immediately reverse transcribed into cDNA using a combination of random decamers and oligo(dT) primers. RT-qPCR was carried out as described below. To examine proteins co-immunoprecipitated with GW182, the washed IP beads were incubated in sample buffer at 95° C. for 5 minutes and subjected to SDS-PAGE electrophoresis and immunoblotting.

RNA Extraction, Northern Blots, Quantitative PCR for Individual mRNA and miRNA Species

Total RNA from primary cultures of mouse hippocampal neurons was isolated by Tri-Reagent (Molecular Research Center, Inc.) according to the manufacturer's protocol. Cultures were either homogenized in Tri-Reagent directly, or were first lysed in lysis buffer plus RNase inhibitor (if protein from the same sample was required) followed by Tri-Reagent addition to a portion of lysate. RNA pellets were air-dried and resuspended in nuclease-free water. RNA concentration and quality were assayed by spectrophotometric measurements at an optical density (OD) 260/280/230 nm.

Northern blots were carried out as described (Hwang et al., 2007), using the following LNA probes from Exiqon: Let-7a (5′ AACTATACAACCTACTACCTCA 3′; SEQ ID NO: 1), pre-Let-7a (5′ GTGGGTGTGACCCTA 3′; SEQ ID NO: 2), miR-17 (5′ CTACCTGCACTGTAAGCACTTTG 3′; SEQ ID NO: 3) and quantified by phosphoimaging.

For analysis of mRNA abundance: 1 μg of RNA was immediately reverse-transcribed into cDNA with a taqman reverse transcripton kit (Applied Biosystems) and a mixture of random decamer and oligo(dT) primers in a final volume of 30 μL and subjected to TaqMan Gene Expression Assays (Applied Biosystems) for GluR1 (assay ID: Mm00433753_m1), CaMKIIu (Mm00437967_m1), Homer2 (Mm01314936_m1), KCC2 (Mm00803929_m1), Kv1.1 (Mm00439977_s1), GW182 (Mm00523487_m1), GAPDH (Mm99999915_g1), and β-tubulin-III (Mm00727586_s1). RT-qPCR was performed using a Stratagene Mx3000P machine and software in 20 μL reactions on a 96-well optical plate at 95° C. for 5 min, followed by 40 cycles of 95° C. for 30 sec, 55° C. for 1 min and 72° C. for 30 sec. The threshold and threshold cycle (Ct) values were determined using default settings. Standard curves were constructed and used for quantitation of target transcript abundance. In this method, 1:5 dilution series of an independent Standard sample are amplified to generate a curve that relates the initial quantity of the specific target in the Standard samples to the Ct. The standard curve is then used to derive by interpolation the initial sample template quantities based on their Ct values. All derived quantities were further normalized to neuron-specific β-tubulin III, whose translational status is unchanged by BDNF (Schratt et al., 2004). Data were plotted as fold change relative to mock control.

For individual microRNA abundance assays (Applied Biosystems), 100 ng of total isolated RNA was prepared for reverse transcription with stem-looped primers specific for individual mature miRNAs in a final volume of 15 μL according to manufacturer's protocol; 4° C. for 5 min, 16° C. for 30 min, 42° C. for 30 min, 85° C. for 5 min. and subjected to TaqMan MicroRNA Assays (Applied Biosystems) for Let-7a (assay ID: 000377), Let-7b (002619), Let-7e (002406), Let-7f1 (000382), miR-107 (000443) and miR-143 (0024). RT-qPCR was performed using a Stratagene Mx3000P machine and software with quantities derived by standard-curve quantitation method. The abundance of non-coding 18s rRNA in each sample was used as an internal control to normalize all miRNA species.

miRNA Profiling Arrays and Analysis

Murine hippocampal cultures were preincubated for 10-20 minutes with 0.5 mg/mL Actinomycin D and either mock- or BDNF- (100 ng/mL) stimulated for 30 minutes before harvesting.

For miRNA arrays, 1 mg of total RNA for each sample was reverse-transcribed with stem-looped Megaplex RT Primers (Applied Biosystems) in a final volume of 7.5 μL according to manufacturer's instructions: preincubation at 4° C. for 5 min; 16° C. for 2 min, 42° C. for 1 min, 50° C. for 1 sec, 40 cycles; 85° C. for 5 min. The entire cDNA RT product (7.5 μk) was subjected to RT-qPCR on an Applied Biosystems 7900HT Fast Real-Time PCR system using Taqman Rodent MicroRNA Array A. Data were compiled in SDS RQ Manager 1.2.1 and analyzed in Data Assist 2.0 (Applied Biosystems); RQ was calculated as 2^{-LCtBDNF}/2^{LCtmock} where LCt=(cycle threshold for miRNA of interest)−(cycle threshold for reference control) (Schmittgen and Livak, 2008). Thresholds and cycle threshold (Ct) values were determined using default settings and the maximum allowable Ct value was set at 35.0 inclusive. Data were normalized by arithmetic mean using MammU6 (4395470) and snoRNA202 (4380914) reference RNAs as controls. miRNA array data are reposited with the NCBI Gene Expression Omnibus (GEO). Initial experiments using an alternative miRNA array platform (Geniom microfluidic miRNA profiling, Febit Inc.) similarly indicated a BDNF-mediated increase in the majority of detectable mature miRNA species.

### Lentivirus Preparation and Gene Knock-Down by RNAi

Lentiviral stocks were prepared as previously described (Lois et al., 2002). Knockdown was by lentiviral-mediated delivery of non-target shRNA (Sigma, SHC002), shRNA targeting GW182 (CCTTAGTAATGGAGAGTCAAA; SEQ ID NO: 4), LSM5 (OpenBiosystems TRCN0000109196), Dicer (TRCN0000071320) or Lin28 (TRCN0000102576) to cultures at multiplicity of infection of 5-10, 48 hours before imaging, or 4-5 days for GW182.

### Luciferase Reporter Assays

The siRNA- or miRNA-reporter constructs harbor one perfectly matched or four bulged CXCR4 siRNA target sites, respectively, in the 3′UTR of firefly luciferase mRNA. In the presence of CXCR4 shRNA, perfectly matched sequences are cleaved by siRISC and bulge-containing sequences are targets for translation suppression by miRISC. miRNA and siRNA pathway function were assayed in cells transiently transfected and expressing either the miRNA or siRNA reporters alone (no sh-CXCR4), or co-expressing either of the reporters and CXCR4 shRNA with or without P-body disruption (sh-Control-1 or GW182KD, GFP or GFP-DNGW182, sh-Control-2 or LSm5 KD).

Let-7 luciferase reporters (gift of G. Hannon) harbor a wildtype or a mutant Let-7 miRNA binding site. The Let-7 miRNA binding sites are derived from a short 3′UTR segment of C. elegans Lin41 mRNA containing two adjacent proven Let-7 miRNA biding sites and are cloned into 3′UTR of firefly luciferase in a pcDNA backbone. Mutations in seed regions of both Let-7 binding sites were made for a negative control reporter, which was documented not regulated by endogenous Let-7 miRNAs.

Co-transfection of the pCSK-lacZ vector, which constitutively expresses β-galactosidase and is not regulated by shRNA, served to normalize transfection efficiency and extract recovery for each sample in all reporter assays. The DNA amounts used for each well (24 well plate) were 15 ng of any luciferase reporter, 85 ng of β-gal (CSK-LacZ), and/or 75-300 ng of CXCR4 shRNA. Each reporter experiment included extracts from cells transfected with pcDNA3.1 alone as a reference control.

44 hours post-transfection, hippocampal cultures were treated with serum-reduced media (0.5% B27) and Actinomycin-D (0.5 ng/mL) as previously described, followed by BDNF stimulation 100 ng/mL for 4 hours. Cell lysates were collected in 1×lysis buffer (reporter lysis buffer, Promega), and luciferase (Promega) and chemiluminescent β-gal (Roche) reporter assays carried out according to manufacturer instructions using a plate-reading luminometer (Perkin Elmer). Samples were compared by subtracting the background activity of the reference control, and then normalizing the luciferase activity of each sample to its β-gal activity (Shrum et al., 2009). When required, fold change was calculated by dividing normalized stimulated samples by normalized unstimulated samples.

### Plasmids and Fluorescently Tagged Constructs

GFP-Dcp1a was a gift from J. Lykke-Andersen (UCSD). GFP-Staufen was a gift from L. DesGroseillers (U. Montreal). YFP-Pat1b was a gift from G. Stoecklin (U. Heidelberg). BFP-Dcp1a was generated by subcloning with EBFP2. GFP-hAgo2 (11590) and GFP-GW182L1 (DNGW182, 11592) were purchased from Addgene.

Cloning of let-7a-1 GGAG Mutant

A let-7a-1 precursor miRNA with the conserved Lin28 “GGAG” recognition motif mutated to “GtAt” was generated by two-step PCR site-directed mutagenesis from the pLV-hsa-let-7a-1 vector (Biosettia, mir-p001) using the following primers: TATAGGATCCTCACACAGGAAACCA (forward, outside; P1; SEQ ID NO: 5) TATAGCTAGCGCTGCACTACATCTC (reverse, outside; P2; SEQ ID NO: 6) CCCACCACTGGTATATAACTATACAATCTACTG (forward, inside; P3; SEQ ID NO: 7) TGTATAGTTATATACCAGTGGTGGGTGTGA (reverse, inside; P4; SEQ ID NO: 8).

The P1 and P4 primers and the P2 and P3 primers were paired for the first round of PCR reactions. The products of these PCR reactions were then included with the P1 and P2 primers for the second round of PCR reactions to generate the final “GtAt” mutant let-7a-1 fragment. This fragment was then subcloned into the BamHI and NheI sites of the parent plv-hsa-let-7a-1 parent vector.

### Statistical and Bioinformatics Analysis

Student's t-tests were performed in Excel. Nonparametric unpaired one-way ANOVA with post hoc Bonferroni analysis, and Mann-Whitney U tests were performed in OpenStat 11.9.08 (Softonic) or STATA 10.0 (StataCorp.).

### Example 2

### Representative Results

It was thought that global regulatory mechanisms for mRNA translation, storage, or degradation might be enlisted to impart specificity to BDNF control of protein synthesis. RNA processing bodies (P-bodies or GW-bodies) are RNA granules that depend upon RNA for their formation (Teixeira et al., 2005), and harbor translationally repressed mRNAs that may be degraded or stored and released for subsequent translation (Brengues et al., 2005). Accordingly, in some embodiments, the presently disclosed data establish that specificity in BDNF-regulated translation depends upon a two-part post-transcriptional control of miRNA biogenesis that generally enhances mRNA repression in association with GW182, while selectively de-repressing and increasing translation of specific mRNAs.

Further, the presently disclosed subject matter demonstrates that BDNF induces the rapid appearance of P-bodies in neurons, and determines that the function of miRNA biogenesis pathways is required for BDNF-mediated regulation of translation as well as the induction of P-bodies. Remarkably, BDNF induces widespread changes in miRNA biosynthesis through enhancement of the general miRNA processing enzyme, Dicer, and elevation of levels of Lin28a, a protein that prevents the processing of a subset of miRNAs. The combined action of BDNF on Dicer and Lin28a mediates target-specificity of BDNF-induced translation by dictating the profile of neuronal miRNAs that target mRNAs for translational repression.

More particularly, as disclosed in more detail herein below, the presently disclosed subject matter demonstrates that BDNF increases levels of Dicer and many mature miRNAs; BDNF also induces Lin28 and selective miRNA loss in terminally differentiated neurons; BDNF-induced dendrite growth requires Lin28-mediated degradation of pre-Let-7 miRNAs; and regulation of miRNA biogenesis underlies BDNF's target selection in protein synthesis.

### A. BDNF Increases Neuronal P-Body Number

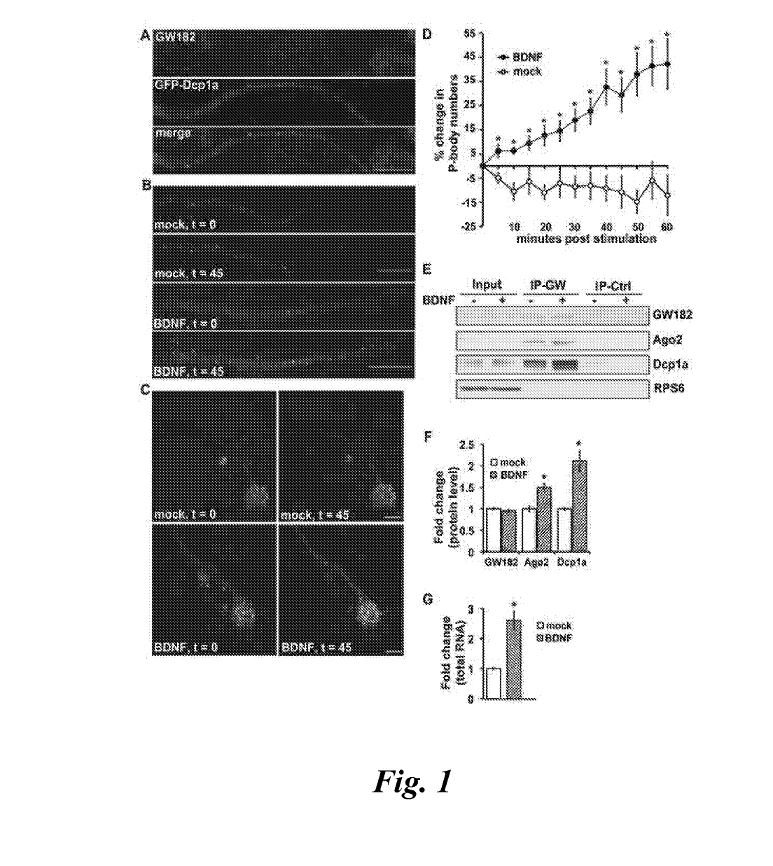

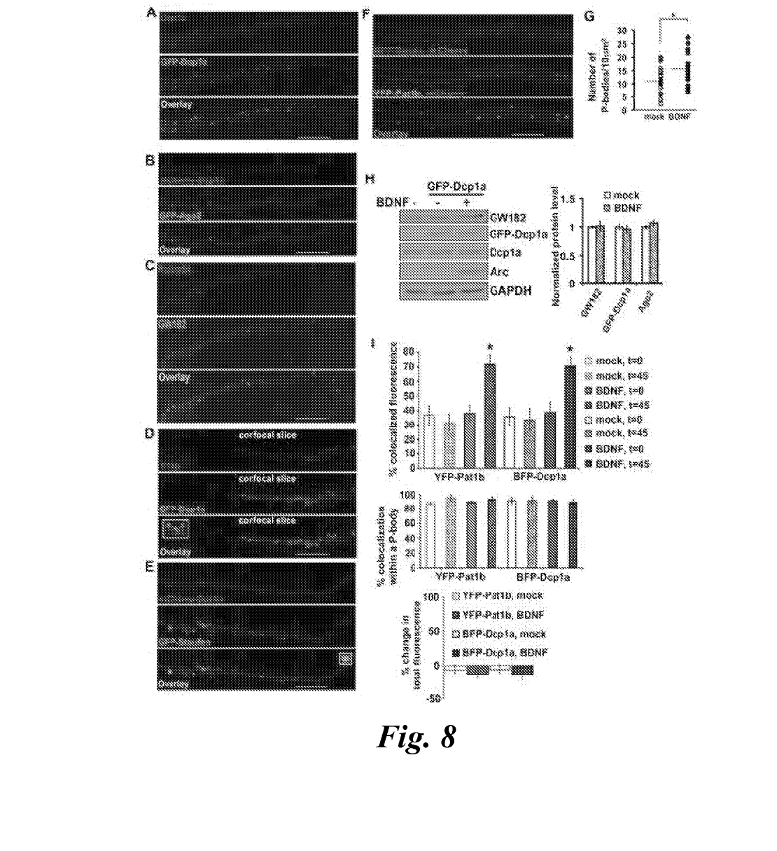

To investigate whether changes in RNA processing might be induced by BDNF, live cell imaging was used to examine BDNF effects on neuronal P-body abundance as a readout of potential broad effects on RNA regulatory mechanisms. P-bodies were monitored by expression of GFP-tagged Dcp1a (GFP-Dcp1a), a decapping enzyme and specific P-body marker (Anderson and Kedersha, 2006) that colocalized with endogenous Dcp1a (FIG. 8A) and other P-body components, including the RNA-binding protein GW182 (neuronal dendrites, see FIG. 1A; FIGS. 8A-F).

BDNF-stimulated hippocampal pyramidal neurons responded with a rapid and robust increase in the number of both dendritic and somatic P-bodies, compared to mock-stimulated neurons, as assessed by live imaging of GFP-Dcp1a (FIGS. 1B-D) or endogenous staining (FIG. 8G). Neurons were pre-incubated and imaged in the presence of the transcription inhibitor, Actinomycin-D, indicating that the rapid increase in P-bodies can be mediated post-transcriptionally. BDNF induces P-body complex formation rather than synthesis of components since protein levels of endogenous Dcp1a or GW182, or GFP-Dcp1a were not altered by BDNF (FIG. 8H), and BDNF enhanced the total co-localization of two tagged P-body components, Dcp1a and Pat1b, without altering their expression (FIGS. 8F,I).

Immunoprecipitation of GW182 demonstrated that BDNF increased the association of P-body components Argonaute 2 (Ago2) and Dcp1a with GW182 (FIG. 1E, F) and, as anticipated since P-bodies require RNA for formation, BDNF induced a more than two-fold increase in the total co-immunoprecipitated RNA (FIG. 1G). Exclusion of ribosomal protein S6 (RPS6) was used to corroborate immunopurification purity (FIG. 1E). Collectively, these data show that the formation of P-bodies, containing non-translating RNA targeted for repression or degradation, is increased by BDNF, a stimulus known to enhance the activity of general translation factors and total cellular translation.

Referring once again generally to FIG. 1, a time-lapse movie (not shown) of P-body response in dendrites of a BDNF-stimulated hippocampal pyramidal neuron expressing mCherry and GFP-Dcp1a indicated occasional movement, but no long-distance translocation of P-bodies, supporting the view that P-bodies primarily form locally rather than being transported from other cellular locations in response to BDNF.

### B. Loss of GW182 Prevents BDNF Regulation of Target Protein Synthesis

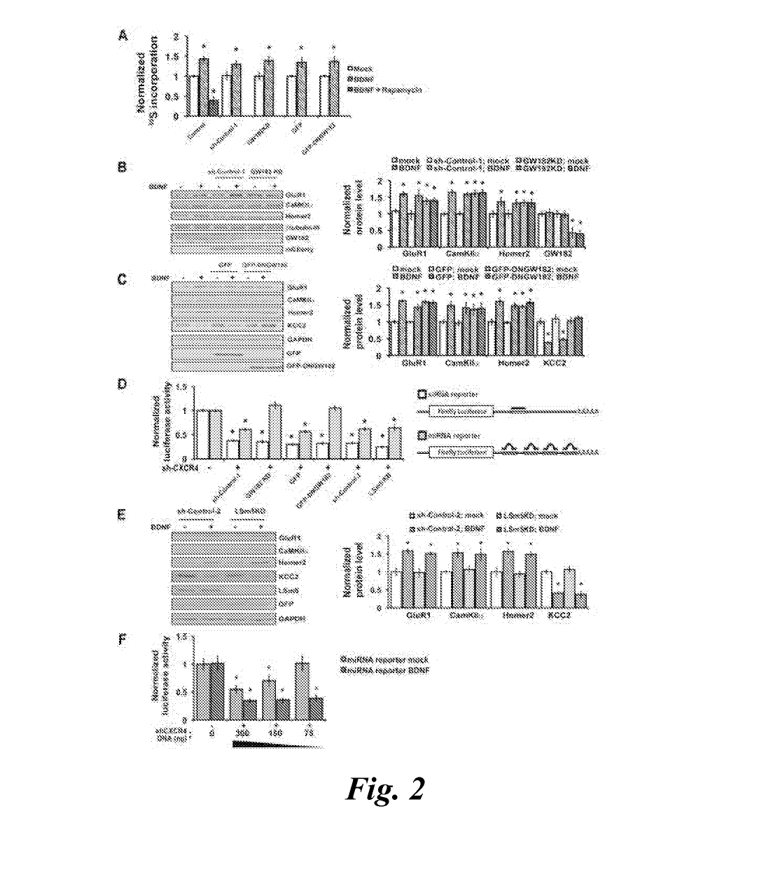

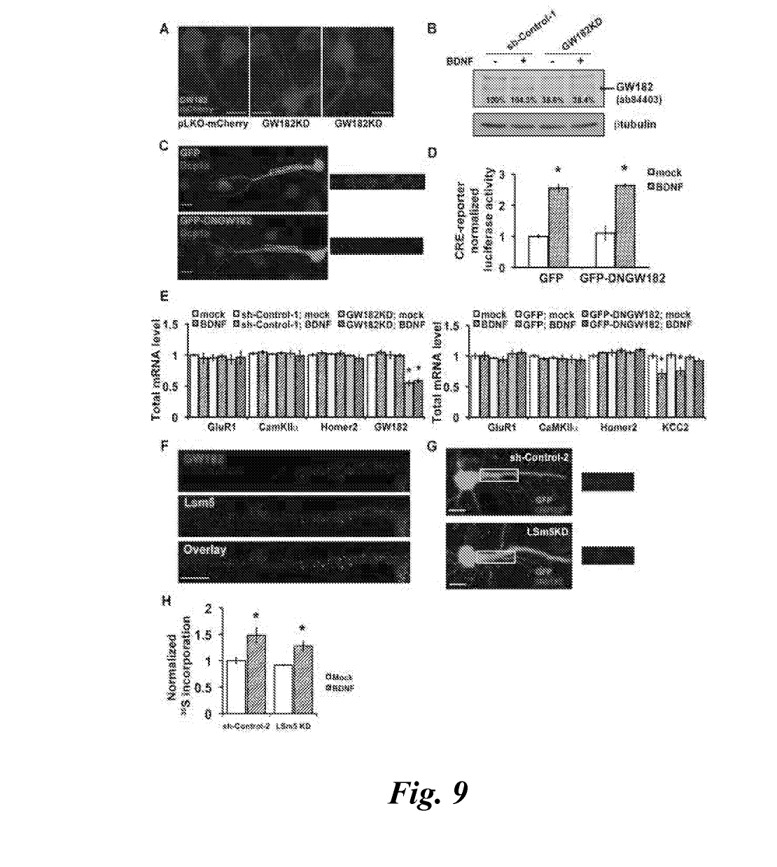

Despite modestly enhancing total cellular translation, BDNF modulation of protein synthesis is highly selective with increases or decreases only in the levels of specific target proteins. To examine whether RNA-processing or repression might factor in this target specificity, the effect of loss of GW182 function was tested by either knockdown of GW182 or by expression of a GFP-tagged dominant negative GW182 (GFP-DNGW182) (Jakymiw et al., 2005). Both manipulations resulted in the loss of visible P-bodies (FIG. 9A-C), as previously reported. Loss of GW182 function did not alter the modest enhancement of total translation mediated by BDNF (FIG. 2A), and also did not interfere with BDNF-regulation of another pathway, CREB-dependent transcription (FIG. 9D).

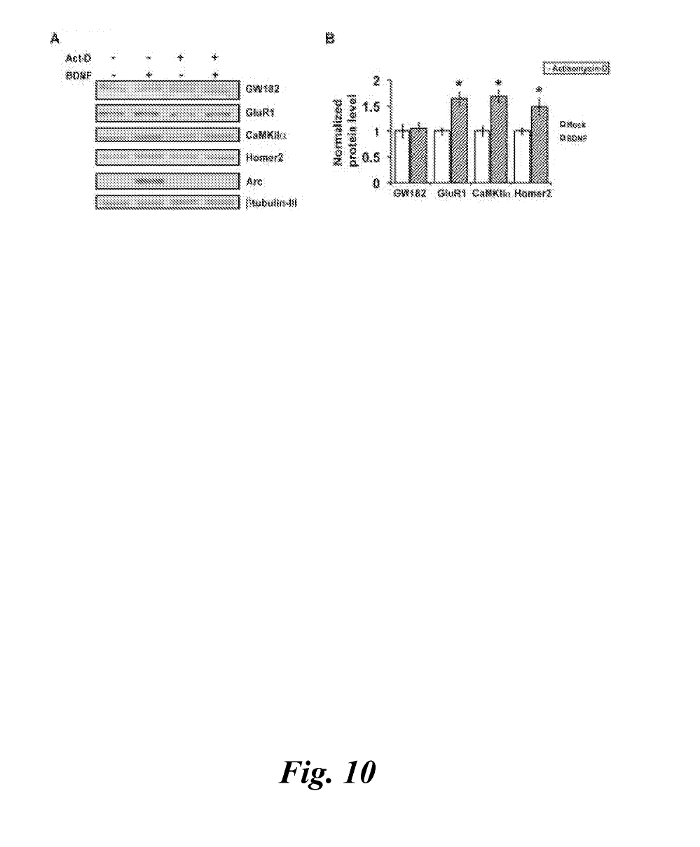

In contrast, GW182 knockdown or DNGW182 expression both strikingly eliminated the mRNA target specificity of BDNF-regulated protein synthesis. The AMPA glutamate receptor subunit GluR1, calcium calmodulin-dependent protein kinase II (CaMKIIu), and Homer2 normally undergo enhanced protein synthesis in response to BDNF (Narisawa-Saito et al., 1999; Schratt et al., 2004), while synthesis of the potassium-chloride co-transporter, KCC2, is decreased by BDNF (Rivera et al., 2002). GW182 knockdown (FIG. 2B) or DNGW182 expression (FIG. 2C) in hippocampal neurons elevated the basal levels of proteins normally upregulated by BDNF (GluR1, CaMKIIu, and Homer2) and prevented their further induction by BDNF. In contrast, the basal protein level of BDNF-downregulated target (KCC2) was unchanged by loss of GW182 function, but KCC2 protein level was no longer reduced by BDNF. β-tubulin III was unchanged by BDNF and used for normalization. These experiments were performed in the presence of Actinomycin-D; similar effects were seen without Actinomycin-D (FIG. 10). Effective GW182 knockdown was achieved using lentiviral transduction and verified by immunoblotting for GW182 (serum 18033, M. Fritzler, FIG. 2B, and Abcam FIG. 9B).

Quantitative real time PCR (RT-qPCR) showed that mRNA levels of BDNF-upregulated targets were unchanged by loss of GW182 function (FIG. 9E), suggesting that the observed changes in basal protein levels could result from altered target translation. As previously reported, BDNF stimulation reduced mRNA levels of the down-regulated target, KCC2, in control neurons (Rivera et al., 2002). In neurons deficient in GW182, however, BDNF no longer significantly reduced the level of KCC2 mRNA (FIG. 9E). These results indicated that GW182 function is required for both baseline translational repression of BDNF-upregulated targets and for BDNF-induced mRNA degradation of a downregulated target. The composite effects implied a role for GW182 in the process that allows BDNF to differentially regulate specific mRNA targets.

C. The Role of miRNA-Mediated Repression in BDNF Regulation of Target Protein Synthesis

Several RNA processing events are associated with P-body formation, including multiple RNA decay mechanisms, mRNA suppression by RNA binding proteins, and RISC-mediated repressive functions. Previous reports demonstrated that P-body disruption through targeting of discrete P-body protein components, such as GW182, can differentially block distinct RNA processing events (Liu et al., 2005a). To test whether functions associated with GW182 in particular were required for translational specificity of BDNF, the effects of loss of GW182 were compared with loss of another P-body component, LSm5. The initial focus centered on assessing RISC-mediated functions, in contrast to decay pathways, since transcript levels of BDNF-upregulated targets appeared unchanged by loss of GW182 (FIG. 9E).

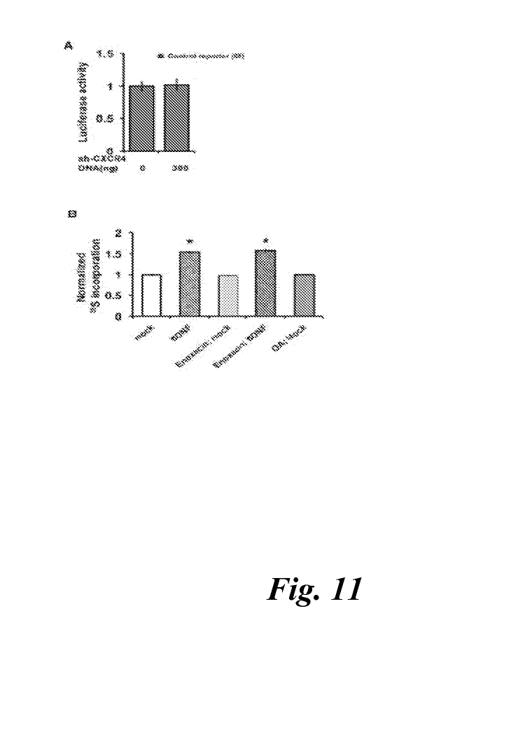

Function of miRNA and siRNA pathways were tested by a reporter assay consisting of co-expression of a hairpin precursor shRNA (shCXCR4) and a luciferase reporter containing 3′UTR binding sites with either perfect (siRNA reporter) or mismatched (miRNA reporter) complementarity for the CXCR4 shRNA (Doench et al., 2003; Wang et al., 2006). Expression of either reporter without the shRNA exhibited full luciferase activity (FIG. 2D); a control reporter lacking CXCR4 binding sites was unaffected by shCXCR4 co-expression (FIG. 11).

P-body disruption by loss of GW182 function produced a preferential miRNA pathway deficit, as shown by failure of coexpressed shRNA to repress the miRNA reporter, with no effect on siRNA-dependent inhibition (FIG. 2D). Loss of GW182 has been previously reported to impair miRNA-mediated translational repression (Jakymiw et al., 2005; Liu et al., 2005a). In contrast, P-body disruption by LSm5 knockdown (FIG. 9F,G) did not significantly alter reporter suppression through siRNA or miRNA pathways in comparison to controls (shRNA-1, shRNA-2, or GFP; FIG. 2D), indicating that these pathways remain intact.

The finding that loss of GW182, but not LSm5, disrupted miRNA-mediated repression, presented the opportunity to probe the importance of miRNA function in determining the specificity of BDNF-regulated translation. In contrast to the loss of specificity in BDNF-regulated protein synthesis produced by GW182 deficiency (FIG. 2B,C), loss of LSm5 did not alter translation specificity (FIG. 2E) even though LSM5 knockdown also disrupted P-bodes (FIG. 9F,G). LSm5 knockdown, like loss of GW182, also did not affect BDNF-enhancement of total cellular translation (FIG. 9H). Comparing the effects of loss of GW182 or LSm5 function suggested the involvement of miRNA-mediated functions in conferring target specificity to BDNF-regulated protein synthesis.

D. Rapid enhancement of mature miRNA biogenesis by BDNF

To further investigate the role of miRNA in the specificity of BDNF-regulated translation, whether BDNF might itself affect the miRNA pathway was investigated.

Intriguingly, BDNF stimulation of cells co-expressing the shCXCR4 and miRNA reporter greatly enhanced miRNA-mediated suppression. Titration of shCXCR4 in this assay revealed that a low dose that did not suppress the reporter in the absence of BDNF generated maximally effective suppression after cellular stimulation with BDNF (FIG. 2F). This effect was independent of new transcription as stimulation was carried out in the presence of Actinomycin-D. CXCR4 shRNA resembles endogenous pre-miRNA and requires Dicer cleavage to generate mature duplex RNA.

It was thought that BDNF could enhance miRNA-mediated repression in the reporter assay by two potential general mechanisms: first, BDNF might increase the efficacy of the RISC complex or, second, BDNF might increase the generation of functional mature duplex miRNA from transfected CXCR4 shRNA. A mechanism invoking BDNF-enhanced mature miRNA biogenesis was congruent with earlier findings since elevated levels of miRNA can deliver additional mRNAs targeted for repression to P-bodies and increase P-body number (Liu et al., 2005b).

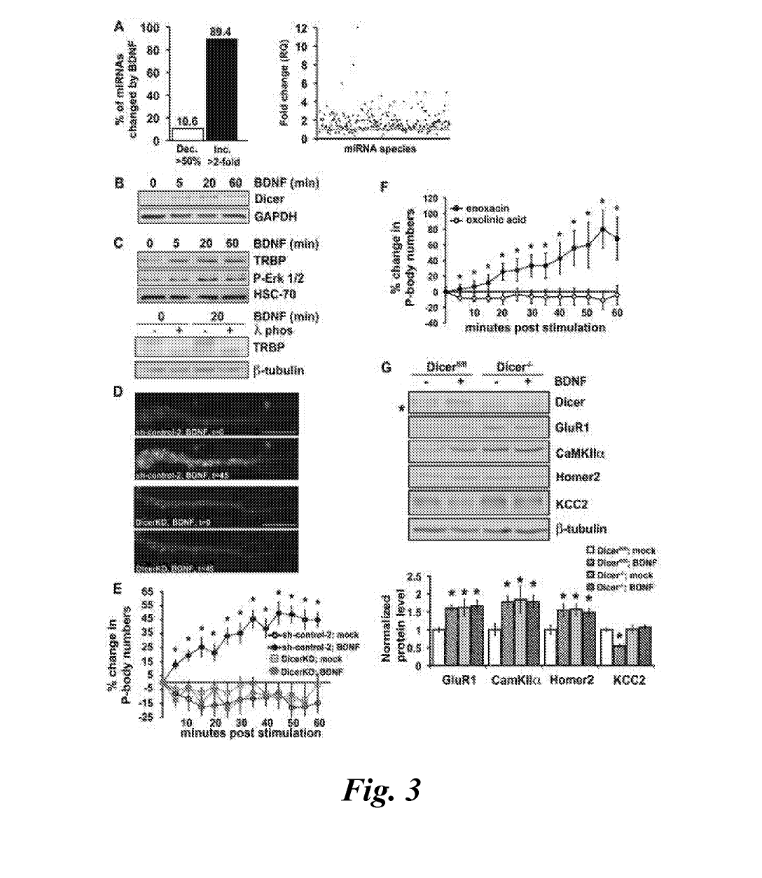

The potential for global regulation of miRNA biogenesis was addressed by BDNF using miRNA arrays that selectively measure mature miRNA, as opposed to pre-miRNA. Hippocampal neurons were treated with BDNF for 30 min in the presence of Actinomycin-D to assess changes due to processing of existing pre-miRNAs rather than new pre-miRNA production. Each array (Taqman) contained 375 rodent miRNA targets of which 195 were detectable in hippocampus in three independent paired experiments. Remarkably, of detectable endogenous miRNAs with levels significantly altered by BDNF, 89.4% were increased more than 2-fold by BDNF, while only 10.6% were decreased to <50% (FIG. 3A, left panel). Many more miRNA species showed smaller, less than 2-fold, posttranscriptional changes in abundance in response to BDNF. While absolute changes in individual miRNAs were not reproducible between paired array experiments, the qualitative effect of a predominantly increased abundance of many miRNA species in response to BDNF was reproducible on this platform as well as an initial analysis using Geniom miRNA biochips (Febit Inc., data not included). An expression analysis of fold change for each detectable miRNA species from the arrays illustrates an overall trend toward higher miRNA quantities in BDNF compared to mock-treated primary neurons (FIG. 3A, right panel).

Widespread post-transcriptional upregulation of mature miRNA production suggested that BDNF might regulate an essential component of miRNA biogenesis, such as the Dicer processing complex. To assess this, Dicer protein levels in BDNF-stimulated neurons were examined BDNF elicited a marked transcription-independent increase in Dicer levels that peaked between 5-20 min after stimulation (FIG. 3B). The binding of BDNF to TrkB receptors triggers signaling pathways promoting growth and survival, including activation of PI3K/AKT and MAPK/ERK pathways. Previous work in tumor cell lines revealed that a component of the Dicer complex, HIV-1 TAR RNA-binding protein (TRBP), could undergo Erk-dependent phosphorylation and that phospho-mimetic TRBP stabilized and enhanced Dicer levels (Paroo et al., 2009); mutations resulting in decreased TRBP protein also destabilize Dicer (Melo et al., 2009). Accordingly, the effect of BDNF on TRBP levels and phosphorylation status in neurons was evaluated. BDNF rapidly induced phospho-ERK and a multiple-banding pattern of TRBP (FIG. 3C, upper panel) that was collapsed by phosphatase treatment (FIG. 3C, lower panel). Total TRBP protein levels were also rapidly elevated and reached significance by 5 min after BDNF (2.61 fold increase +/−0.86).

To evaluate a requirement for Dicer activity and miRNA biogenesis in BDNF-induced recruitment of non-translating mRNA to P-bodies, Dicer was depleted by RNAi. Dicer knockdown completely prevented a BDNF-induced increase in P-body numbers (FIG. 3 D, E) in hippocampal pyramidal neurons. Neurons expressing control non-target shRNA responded to BDNF similarly to wildtype neurons (FIG. 3E, FIG. 1D). The requirement for Dicer in BDNF induction of P-bodies indicated a role for Dicer in targeting some mRNAs to the non-translating pool in response to BDNF.

Whether Dicer activation, and by implication an increase in mature miRNAs, was sufficient to generate P-bodies in neurons was investigated next. The fluoroquinolone, enoxacin, was previously shown to promote pre-miRNA processing by the Dicer/TRBP complex while a structurally similar derivative, oxolinic acid, did not significantly increase miRNA biogenesis (Shan et al., 2008). Enoxacin, but not equimolar oxolinic acid, rapidly and robustly increased P-body numbers in neuronal soma and dendrites (FIG. 3F). In comparison to BDNF, the time course of P-body induction by enoxacin was slightly more rapid, consistent with a more direct signaling mechanism. In accordance with a role for miRNA in regulating mRNA target selection, but not bulk translation, enoxacin did not alter basal or BDNF-induced total protein synthesis (FIG. 11B). These results defined a potential mechanism for regulation of miRNA biogenesis by BDNF and linked BDNF-upregulation of Dicer activity to rapid changes in mRNA repression.

To test whether Dicer also is required for the regulation of BDNF-target genes, the response of representative up- and down-regulated BDNF targets was examined in the presence or absence of Dicer. Hippocampal pyramidal neurons from mice with a conditional Dicer allele (Dicer^{flox/flox}, 3A8 line) (Andl et al., 2006) were infected with lentivirus expressing 4-hydroxy tamoxifen (OHT)-inducible Cre recombinase and subsequently mock- or BDNF-stimulated with or without OHT. Targets that are normally low at baseline and upregulated by BDNF, including GluR1, CaMKIIu, and Homer2, were each elevated at baseline in Dicer-deficient neurons, consistent with basal de-repression in the absence of Dicer, and failed to be further upregulated by BDNF. A representative target normally downregulated by BDNF, KCC2, was non-responsive to BDNF in Dicer-deficient neurons (FIG. 3G). Collectively, these results demonstrate that BDNF lacks specificity for up- or down-regulated targets in the absence of Dicer, consistent with a critical role for Dicer in BDNF-induced sequestration of mRNAs in P-bodies and in the mechanism determining the selective regulation of target mRNAs by BDNF.

E. BDNF Confers Selectivity to miRNA Biogenesis Through Lin28a

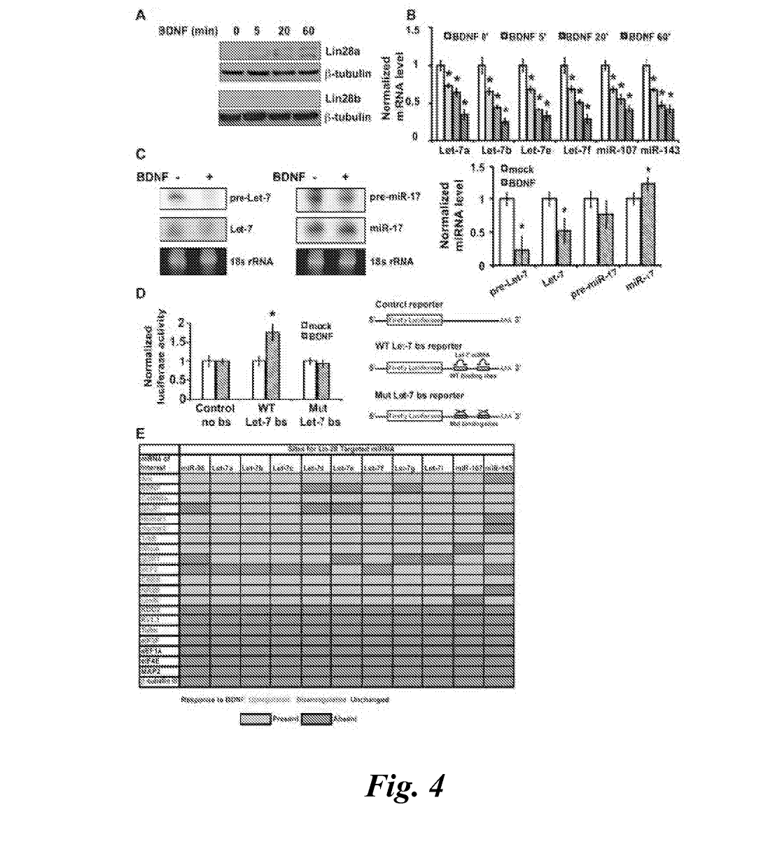

Widespread upregulation of miRNA production and consequent removal of mRNAs from the translating pool by targeted repression provides a viable negative selection mechanism to account for the low proportion of mRNAs reported to undergo BDNF-enhanced translation (Schratt et al., 2004). Whether the miRNA biogenesis pathway might also be regulated to generate positive selection of BDNF-upregulated targets in protein synthesis was investigated. By miRNA array analysis, a small number of miRNAs were observed to decrease in response to BDNF; the decreases were more apparent in some individual experiments than in the collective averaged array data. Among these decreased miRNAs were several members of the Let-7 family. miRNA biogenesis can be regulated at multiple steps by trans-acting factors, including the Lin28 RNA-binding proteins (Heo et al., 2009; Newman et al., 2008; Viswanathan et al., 2008) which target Let-7 family members. Lin28 binding and subsequent pre-miRNA uridylation suppresses processing of targeted pre-miRNA to mature miRNA (Hagan et al., 2009; Heo et al., 2009), and could provide a mechanism for decreasing select mature miRNAs even in the context of Dicer elevation. Consistent with this possibility, a robust and rapid transcription-independent increase in Lin28a, but not Lin28b, protein in mature neurons by 5 min following BDNF exposure was found (FIG. 4A).

Analysis of the terminal loop region of the Let-7, miR-107, and miR-143 pre-miRNAs showed that each has a putative or previously functionally confirmed ‘GGAG’ sequence motif that can permit recognition by Lin28(Hagan et al., 2009; Heo et al., 2009). Individual RT-qPCR assays showed significant and reproducible BDNF-induced decreases in abundance of all tested members of the Let-7 family, as well as miR-107, and miR-143 (FIG. 4B), even though not all were reproducibly detected through the less sensitive miRNA arrays. Significant decreases in each of these miRNAs were apparent by 5 min post-BDNF stimulation (FIG. 4B). In accordance with the expected effects of Lin28, Northern blotting for a member of the Let-7 family (Let7-a) showed significant decreases in both pre-Let7 and mature Let-7 miRNA levels in BDNF-treated neurons (FIG. 4C). A control mature miRNA, miR-17, is modestly increased by BDNF, consistent with enhanced Dicer processing (FIG. 4C).

If Lin28 positively selects BDNF-upregulated targets by decreasing specific miRNAs, an mRNA containing functional binding sites for a Lin28-down-regulated miRNA would be predicted to undergo BDNF-enhanced translation. To test this prediction, the response to BDNF of luciferase reporters whose 3′UTR contained either wildtype or mutated Let-7 miRNA binding sites, or no miRNA binding sites, were compared under conditions of transcription blockade. As predicted, the reporter containing Let-7 binding sites was significantly induced by BDNF, while levels of the control reporters were unchanged (FIG. 4D). This result indicates that downregulation of Let7 family members by BDNF is sufficient to relieve repression and mediate positive target selection for BDNF-enhanced translation.

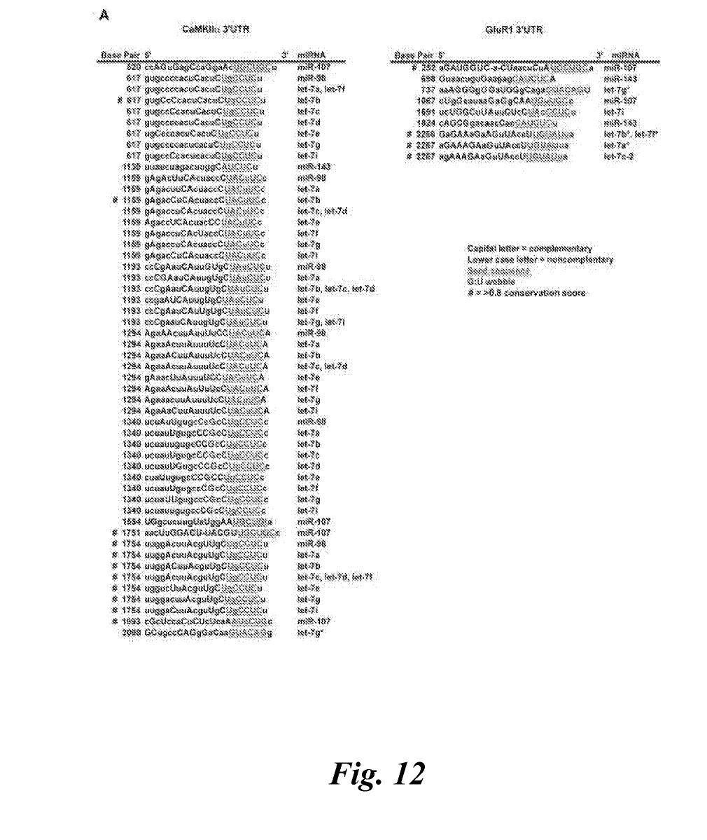

To examine the extent to which this mechanism could generalize to known BDNF targets, the presence of binding sites for Lin28-regulated miRNAs in the 3′UTRs of mRNAs known to undergo upregulated, downregulated, or unchanged translation in response to BDNF were examined Positive scores (light gray boxes, (FIG. 4E)) were restricted to sites in which the miRNA seed sequence (nucleotides 2-7) paired as a perfect or G-U wobble-containing match; similar miRNA seed sequence pairing was previously found important for target recognition (Guo et al., 2010). Thirteen representative BDNF-upregulated targets were all found to contain two or more sites for a Lin28-regulated miRNA (example sites in FIG. 12), while targets known to be downregulated by BDNF (KCC2, KV 1.1) (Raab-Graham et al., 2006; Rivera et al., 2002) or unregulated by BDNF (eEF1A, eIF4E, MAP2, β-tubulin III) (Schratt et al., 2004; Wang et al., 2009) did not contain such sites (FIG. 4E).

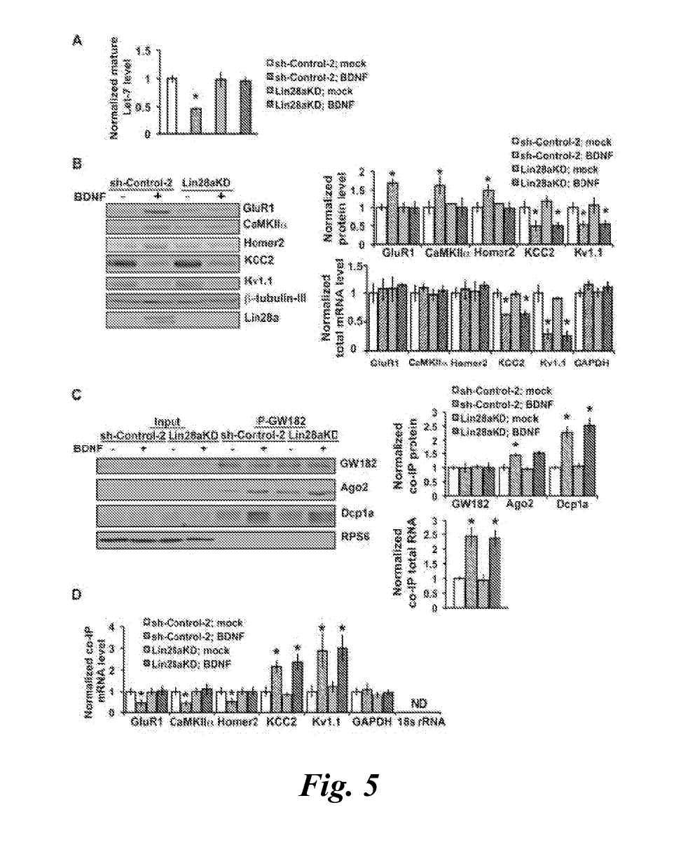

The role of Lin28a in BDNF target mRNA selection was then directly tested. Depletion of Lin28a through RNAi, but not expression of a control hairpin, prevented the decline in mature Let-7 miRNAs in hippocampal neurons responding to BDNF (FIG. 5A). Lin28a knockdown also prevented the increased translation of representative mRNA targets normally upregulated by BDNF (FIG. 5B). In accordance with basal mRNA repression by miRNAs that are diminished through Lin28, protein levels of these normally upregulated targets (CaMKIIu, GluR1, and Homer2) remained at low basal levels even in the presence of BDNF under Lin28 knockdown conditions (FIG. 5B, left and top right panels). Lin28a knockdown did not prevent the increased association of P-body protein and RNA components in response to BDNF (FIG. 5C), which instead requires Dicer (FIG. 3).

Targets normally de-repressed and upregulated by BDNF remained repressed in Lin28a-deficient neurons, in contrast with the effects of loss of GW182 (FIG. 2B,C) or Dicer (FIG. 3G) which both resulted in de-repression of BDNF-induced targets at baseline and occlusion of further upregulation by BDNF. Notably, targets normally downregulated by BDNF, represented by KCC2 and KV1.1, remained responsive to BDNF in the presence of Lin28a knockdown. BDNF effects on target mRNA levels were unaffected by loss of Lin28a (FIG. 5B, bottom right panel). These findings are consistent with translation specificity in response to BDNF being generated by a two-part regulation of the miRNA biogenesis pathway: (1) general upregulation of miRNA biogenesis that is required for repression of mRNAs whose protein products are decreased in response to BDNF; and (2) downregulation of select miRNAs whose processing is blocked by the BDNF-induced Lin28a, resulting in de-repression and enhanced translation of mRNAs containing binding sites for Lin28-regulated miRNAs.

The role of Lin28a in BDNF-regulated translation by evaluating its effects on specific target mRNA repression in association with GW182 was further examined GW182-associated RNA was immunopurified, as in FIG. 1E, under control shRNA and Lin28 knockdown conditions and the effects of BDNF on mRNA recruitment to GW182 were assessed by individual RT-qPCR assays. mRNAs undergoing regulated translation were enriched in overall association with GW182 in comparison to a ‘housekeeping’ GAPDH mRNA (FIG. 5D). Under control shRNA conditions, BDNF reduced the GW182-association of representative mRNAs whose translation is upregulated by BDNF (GluR1, CaMKIIu, and Homer2); in contrast, BDNF promoted the GW182-association of representative mRNAs (KCC2 and Kv1.1) whose translation is downregulated by BDNF (FIG. 5D). β-tubulin III mRNA translation is unchanged by BDNF (Schratt et al., 2004) and was used for normalization; 18s rRNA is absent from P-bodies and served as a control for immunopurification purity (FIG. 5D).

As expected, if Lin28 regulates only selection of BDNF-upregulated targets, Lin28 knockdown altered only the GW182 enrichment profile of representative mRNAs (CaMKIIu, GluR1, and Homer2) that undergo BDNF-enhanced translation (FIG. 5D). mRNAs for BDNF-upregulated targets remained equivalently repressed and associated with GW182 in Lin28a knockdown neurons in the presence or absence of BDNF, and protein levels of these targets were no longer enhanced by BDNF. In contrast, mRNAs for representative BDNF-downregulated targets (KCC2 and Kv1.1) remained enriched in association with GW182 at baseline and their enrichment was equivalently increased by BDNF in both control and Lin28 knockdown neurons (FIG. 5D). A target not regulated by BDNF (GAPDH) was not enriched in GW182-association at baseline, did not change in response to BDNF, and also was unaffected by Lin28 loss (FIG. 5D). These findings indicate that Lin28a, induced by BDNF, is required to suppress the processing of specific pre-miRNAs and selectively decrease levels of these mature miRNAs, concomitant with a general BDNF-induced upregulation in the biogenesis of most miRNAs by enhanced Dicer levels. The negative regulation of miRNA biogenesis by Lin28 presents a mechanism for the selection of upregulated targets in BDNF-induced protein synthesis.

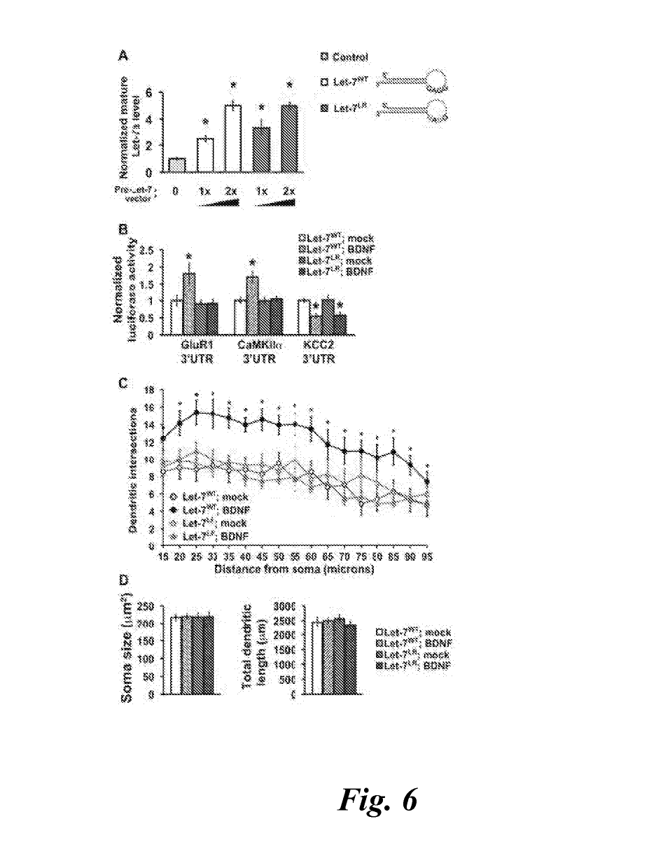

To further test the mechanism by which Lin28 mediates induced translation of BDNF-upregulated targets, a Let-7 pre-miRNA was constructed that would be resistant to Lin28-mediated degradation through mutation of the pre-miRNA terminal loop residues from GGAG to GUAU. This mutation prevents Lin28-induced uridinylation and degradation of Let-7 pre-miRNA (Heo et al., 2009), but does not alter the target specificity of the Let-7 miRNA. Lentiviral-mediated expression of either wildtype (Let-7^{WT}) or Lin28-resistant Let-7 (Let-7^{LR}) in hippocampal cultures enhanced mature Let-7 levels in a dose-dependent manner that could be titrated to achieve equivalent and low levels of exogenous Let-7 expression (FIG. 6A). Co-expression of Let-7^{LR} with reporters harboring the 3′UTR from either of two BDNF-upregulated targets (GluR1 or CaMKIIu) completely prevented their induction by BDNF. In contrast, co-expression of Let-7^{LR} with a reporter harboring the 3′UTR from a BDNF-downregulated target (KCC2) had no effect on the BDNF-mediated depression of this reporter (FIG. 6B). These results supported a selective role for Lin28 in mediating the specificity of BDNF for its upregulated targets.

Therefore, in some embodiments, the presently disclosed subject matter provides an expression construct for Lin28-resistant Let-7 pre-microRNA, which is suitable for mammalian expression of Lin28-resistant Let-7 pre-microRNA. In one embodiment, the presently disclosed construct is in a lentiviral backbone, i.e., a lentiviral vector, making production of active lentiviral particles possible, therefore also allowing effective expression even in cells having low transfection efficiency. In other embodiments, the presently disclosed Lin28-resistant Let-7 pre-microRNA is cloned into other vectors that allow expression in cells, such as an adenovirus based vector and the like.

While alternative mechanisms for selectivity could co-exist, the collective results strongly indicate that dual control by BDNF of the miRNA biogenesis pathway through Lin28a and Dicer critically contributes to determining both up- and down-regulated target specificity in BDNF-mediated protein synthesis. These findings prompted an investigation of the effects of loss of Lin28a on a physiological response requiring BDNF-regulated protein synthesis.

F. Loss of miRNA-Mediated Regulation Prevents BDNF-Enhanced Dendrite Arborization

Induction of dendrite outgrowth in excitatory neurons, both in culture and in-vivo, is a well-characterized BDNF function requiring the regulation translation (Jaworski et al., 2005). Since inhibiting new translation blocks BDNF-induction of dendrite growth, it was thought that BDNF-upregulated targets, selected by Lin28, might be particularly important for this process. The physiological relevance of selective mRNA translation by release from Lin28-targeted miRNAs was tested using low-dose BDNF to stimulate proximal dendrite growth in developing hippocampal pyramidal neurons expressing Let-7^{LR}, or Let-7^{WT} as a control. Based on results disclosed herein (FIGS. 5, 6A,B) and the distribution of sites for Lin-28 targeted miRNAs (FIGS. 4E, 12), Let-7^{LR} expression could be expected to function as a dominant negative to repress mRNA targets despite BDNF-mediated elevation of Lin28.

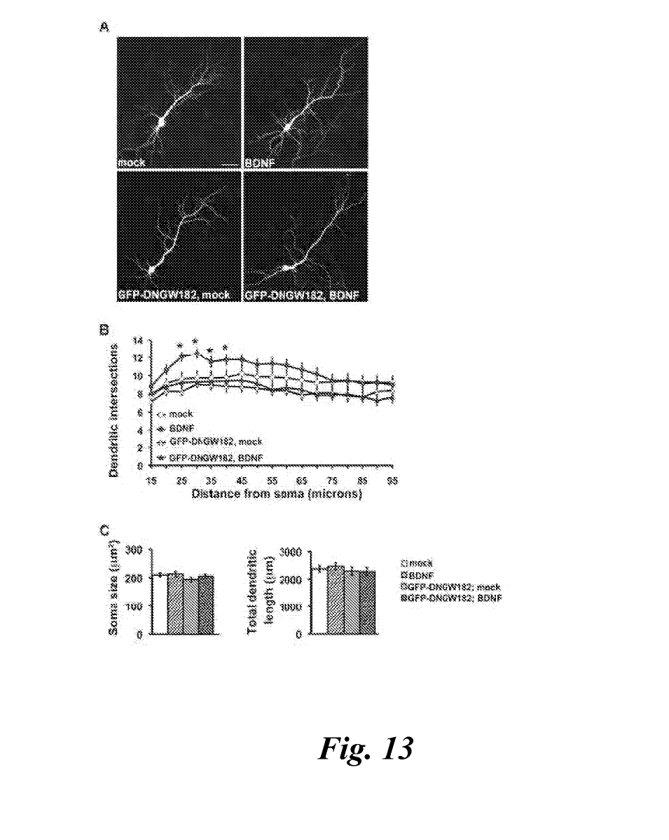

Analysis of dendrite complexity (supplemental information), showed that Let-7^{LR} expression prevented BDNF enhancement of dendrite outgrowth (FIG. 6C), without significantly altering basal dendrite complexity (mock condition) in comparison to control neurons expressing either Let-7^{WT} (FIG. 6C) or GFP (FIG. 13B, p=0.78, one-way ANOVA), or cell soma size and total dendritic length, which were also unaffected by BDNF (FIG. 6D). Loss of GW182 function, which would be expected to inhibit miRNA-mediated repression by both Lin28regulated and non-Lin28-regulated miRNAs, also prevented BDNF-induced dendrite growth without altering basal dendrite complexity or total protein synthesis (FIGS. 13, 2A). These experiments highlight the importance of miRNA-mediated target selection in a neuronal response to BDNF requiring the induction of protein synthesis. In sum, it appears that Lin28-induced degradation of pre-miRNAs is specifically required for the appropriate specification of mRNA targets for BDNF-upregulated translation and is required for BDNF-dependent growth of neuronal dendrites.

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art.
Aakalu, G., Smith, W. B., Nguyen, N., Jiang, C., and Schuman, E. M. (2001). Dynamic visualization of local protein synthesis in hippocampal neurons. Neuron 30, 489-502.
Anderson, P., and Kedersha, N. (2006). RNA granules. J Cell Biol 172, 803-808.
Andl, T., Murchison, E. P., Liu, F., Zhang, Y., Yunta-Gonzalez, M., Tobias, J. W., Andl, C. D., Seykora, J. T., Hannon, G. J., and Millar, S. E. (2006). The miRNA-processing enzyme dicer is essential for the morphogenesis and maintenance of hair follicles. Curr Biol 16, 1041-1049.
Aoto, J., Nam, C. I., Poon, M. M., Ting, P., and Chen, L. (2008). Synaptic signaling by all-trans retinoic acid in homeostatic synaptic plasticity. Neuron 60, 308-320.
Brengues, M., Teixeira, D., and Parker, R. (2005). Movement of eukaryotic mRNAs between polysomes and cytoplasmic processing bodies. Science 310, 486-489.
Chendrimada, T. P., Finn, K. J., Ji, X., Baillat, D., Gregory, R. I., Liebhaber, S. A., Pasquinelli, A. E., and Shiekhattar, R. (2007). MicroRNA silencing through RISC recruitment of eIF6. Nature 447, 823-828.
Doench, J. G., Petersen, C. P., and Sharp, P. A. (2003). siRNAs can function as miRNAs. Genes Dev 17, 438-442.
Eulalio, A., Behm-Ansmant, I., Schweizer, D., and Izaurralde, E. (2007). P-body formation is a consequence, not the cause, of RNA-mediated gene silencing. Mol Cell Biol 27, 3970-3981.
Guo, H., Ingolia, N. T., Weissman, J. S., and Bartel, D. P. (2010). Mammalian microRNAs predominantly act to decrease target mRNA levels. Nature 466, 835-840.
Hagan, J. P., Piskounova, E., and Gregory, R. I. (2009). Lin28 recruits the TUTase Zcchc11 to inhibit let-7 maturation in mouse embryonic stem cells. Nature structural & molecular biology 16, 1021-1025.
Hendrickson, D. G., Hogan, D. J., McCullough, H. L., Myers, J. W., Herschlag, D., Ferrell, J. E., and Brown, P. O. (2009). Concordant regulation of translation and mRNA abundance for hundreds of targets of a human microRNA. PLoS biology 7, e1000238.
Heo, I., Joo, C., Kim, Y. K., Ha, M., Yoon, M. J., Cho, J., Yeom, K. H., Han, J., and Kim,
V. N. (2009). TUT4 in concert with Lin28 suppresses microRNA biogenesis through premicroRNA uridylation. Cell 138, 696-708.

Huber, K. M., Kayser, M. S., and Bear, M. F. (2000). Role for rapid dendritic protein synthesis in hippocampal mGluR-dependent long-term depression. Science 288, 12541257.
Hwang, H. W., Wentzel, E. A., and Mendell, J. T. (2007). A hexanucleotide element directs microRNA nuclear import. Science 315, 97-100.
Jakymiw, A., Lian, S., Eystathioy, T., Li, S., Satoh, M., Hamel, J. C., Fritzler, M. J., and Chan, E. K. (2005). Disruption of GW bodies impairs mammalian RNA interference. Nat Cell Biol 7, 1267-1274.
Jaworski, J., Spangler, S., Seeburg, D. P., Hoogenraad, C. C., and Sheng, M. (2005). Control of dendritic arborization by the phosphoinositide-3′-kinase-Akt-mammalian target of rapamycin pathway. J Neurosci 25, 11300-11312.
Keene, J. D., Komisarow, J. M., and Friedersdorf, M. B. (2006). RIP-Chip: the isolation and identification of mRNAs, microRNAs and protein components of ribonucleoprotein complexes from cell extracts. Nature protocols 1, 302-307.
Kertesz, M., Iovino, N., Unnerstall, U., Gaul, U., and Segal, E. (2007). The role of site accessibility in microRNA target recognition. Nature genetics 39, 1278-1284.
Krol, J., Busskamp, V., Markiewicz, I., Stadler, M. B., Ribi, S., Richter, J., Duebel, J., Bicker, S., Fehling, H. J., Schubeler, D., et al. (2010). Characterizing light-regulated retinal microRNAs reveals rapid turnover as a common property of neuronal microRNAs. Cell 141, 618-631.
Liu, J., Rivas, F. V., Wohlschlegel, J., Yates, J. R., 3rd, Parker, R., and Hannon, G. J. (2005a). A role for the P-body component GW182 in microRNA function. Nat Cell Biol 7, 1261-1266.
Liu, J., Valencia-Sanchez, M. A., Hannon, G. J., and Parker, R. (2005b). MicroRNAdependent localization of targeted mRNAs to mammalian P-bodies. Nat Cell Biol 7, 719-723.
Lois, C., Hong, E. J., Pease, S., Brown, E. J., and Baltimore, D. (2002). Germline transmission and tissue-specific expression of transgenes delivered by lentiviral vectors. Science 295, 868-872.
Lugli, G., Torvik, V. I., Larson, J., and Smalheiser, N. R. (2008). Expression of microRNAs and their precursors in synaptic fractions of adult mouse forebrain. J Neurochem 106, 650-661.
Mathonnet, G., Fabian, M. R., Svitkin, Y. V., Parsyan, A., Huck, L., Murata, T., Biffo, S., Merrick, W. C., Darzynkiewicz, E., Pillai, R. S., et al. (2007). MicroRNA inhibition of translation initiation in vitro by targeting the cap-binding complex eIF4F. Science 317, 1764-1767.
Meffert, M. K., Chang, J. M., Wiltgen, B. J., Fanselow, M. S., and Baltimore, D. (2003). NF-kappa B functions in synaptic signaling and behavior. Nat Neurosci 6, 1072-1078.
Melo, S. A., Ropero, S., Moutinho, C., Aaltonen, L. A., Yamamoto, H., Calin, G. A., Rossi, S., Fernandez, A. F., Carneiro, F., Oliveira, C., et al. (2009). A TARBP2 mutation in human cancer impairs microRNA processing and DICER1 function. Nature genetics 41, 365-370.
Moser, J. J., Chan, E. K., and Fritzler, M. J. (2009). Optimization of immunoprecipitation-western blot analysis in detecting GW182-associated components of GW/P bodies. Nature protocols 4, 674-685.
Moss, E. G., and Tang, L. (2003). Conservation of the heterochronic regulator Lin-28, its developmental expression and microRNA complementary sites. Developmental biology 258, 432-442.
Narisawa-Saito, M., Carnahan, J., Araki, K., Yamaguchi, T., and Nawa, H. (1999). Brain-derived neurotrophic factor regulates the expression of AMPA receptor proteins in neocortical neurons. Neuroscience 88, 1009-1014.
Newman, M. A., Thomson, J. M., and Hammond, S. M. (2008). Lin-28 interaction with the Let-7 precursor loop mediates regulated microRNA processing. Rna 14, 1539-1549.
Paroo, Z., Ye, X., Chen, S., and Liu, Q. (2009). Phosphorylation of the human microRNA-generating complex mediates MAPK/Erk signaling. Cell 139, 112-122.
Petersen, C. P., Bordeleau, M. E., Pelletier, J., and Sharp, P. A. (2006). Short RNAs repress translation after initiation in mammalian cells. Mol Cell 21, 533-542.

Raab-Graham, K. F., Haddick, P. C., Jan, Y. N., and Jan, L. Y. (2006). Activity- and mTOR-dependent suppression of Kv1.1 channel mRNA translation in dendrites. Science 314, 144-148.

Rivera, C., Li, H., Thomas-Crusells, J., Lahtinen, H., Viitanen, T., Nanobashvili, A., Kokaia, Z., Airaksinen, M. S., Voipio, J., Kaila, K., et al. (2002). BDNF-induced TrkB activation down-regulates the K+—Cl− cotransporter KCC2 and impairs neuronal Cl− extrusion. The Journal of cell biology 159, 747-752.
Schmittgen, T. D., and Livak, K. J. (2008). Analyzing real-time PCR data by the comparative C(T) method. Nature protocols 3, 1101-1108.
Schratt, G. M., Nigh, E. A., Chen, W. G., Hu, L., and Greenberg, M. E. (2004). BDNF regulates the translation of a select group of mRNAs by a mammalian target of rapamycin-phosphatidylinositol 3-kinase-dependent pathway during neuronal development. J Neurosci 24, 7366-7377.
Sethi, P., and Lukiw, W. J. (2009). Micro-RNA abundance and stability in human brain: specific alterations in Alzheimer's disease temporal lobe neocortex. Neurosci Lett 459, 100-104.
Shan, G., Li, Y., Zhang, J., Li, W., Szulwach, K. E., Duan, R., Faghihi, M. A., Khalil, A. M., Lu, L., Paroo, Z., et al. (2008). A small molecule enhances RNA interference and promotes microRNA processing. Nat Biotechnol 26, 933-940.
Shrum, C. K., Defrancisco, D., and Meffert, M. K. (2009). Stimulated nuclear translocation of NF-kappaB and shuttling differentially depend on dynein and the dynactin complex. Proc Natl Acad Sci USA 106, 2647-2652.
Takei, N., Kawamura, M., Ishizuka, Y., Kakiya, N., Inamura, N., Namba, H., and Nawa, H. (2009). Brain-derived neurotrophic factor enhances the basal rate of protein synthesis by increasing active eukaryotic elongation factor 2 levels and promoting translation elongation in cortical neurons. J Biol Chem 284, 26340-26348.
Teixeira, D., Sheth, U., Valencia-Sanchez, M. A., Brengues, M., and Parker, R. (2005). Processing bodies require RNA for assembly and contain nontranslating mRNAs. Rna 11, 371-382.
Viswanathan, S. R., Daley, G. Q., and Gregory, R. I. (2008). Selective blockade of microRNA processing by Lin28. Science 320, 97-100.
Viswanathan, S. R., Powers, J. T., Einhorn, W., Hoshida, Y., Ng, T. L., Toffanin, S., O'Sullivan, M., Lu, J., Phillips, L. A., Lockhart, V. L., et al. (2009). Lin28 promotes transformation and is associated with advanced human malignancies. Nature genetics 41, 843-848.
Wang, B., Love, T. M., Call, M. E., Doench, J. G., and Novina, C. D. (2006). Recapitulation of short RNA-directed translational gene silencing in vitro. Mol Cell 22, 553-560.

Wang, D. O., Kim, S. M., Zhao, Y., Hwang, H., Miura, S. K., Sossin, W. S., and Martin, K. C. (2009). Synapse- and stimulus-specific local translation during long-term neuronal plasticity. Science 324, 1536-1540.
Yin, Y., Edelman, G. M., and Vanderklish, P. W. (2002). The brain-derived neurotrophic factor enhances synthesis of Arc in synaptoneurosomes. Proc Natl Acad Sci USA 99, 2368-2373.
Yu, J., Vodyanik, M. A., Smuga-Otto, K., Antosiewicz-Bourget, J., Frane, J. L., Tian, S., Nie, J., Jonsdottir, G. A., Ruotti, V., Stewart, R., et al. (2007). Induced pluripotent stem cell lines derived from human somatic cells. Science 318, 1917-1920.

Although the foregoing subject matter has been described in some detail by way of illustration and example for purposes of clarity of understanding, it will be understood by those skilled in the art that certain changes and modifications can be practiced within the scope of the appended claims.

The claims are as follows:

1. A precursor microRNA molecule, the precursor microRNA molecule comprising a nucleic acid comprising:
(a) a precursor microRNA sequence; and
(b) a mutation in the sequence that prevents a protein involved in regulating developmental timing, oncogenesis, and/or neuronal growth from blocking processing of the precursor microRNA sequence to a mature microRNA.

2. The molecule of claim 1, wherein the precursor microRNA sequence is Let-7 (SEQ ID NO: 12).

3. The molecule of claim 1, wherein the protein involved in regulating developmental timing, oncogenesis, and/or neuronal growth is Lin28.

4. The molecule of claim 1, wherein the mutation is in the part of the precursor microRNA sequence that forms a terminal loop structure.

5. The molecule of claim 4, wherein the mutation is a change from the nucleotide sequence GGAG to the nucleotide sequence GUAU.

6. A DNA molecule encoding the precursor microRNA molecule of claim 1.

7. The DNA molecule of claim 6, wherein the DNA molecule encoding the precursor microRNA molecule is Let-7 (SEQ ID NO: 10).

8. The DNA molecule of claim 6, wherein the DNA molecule is operably linked to a recombinant expression vector.

9. The DNA molecule of claim 8, wherein the vector is a lentiviral vector.

10. A kit comprising the DNA molecule of claim 6.

11. A cell comprising one or more of:
(a) a precursor microRNA molecule, the precursor microRNA molecule comprising a nucleic acid comprising:
(i) a precursor microRNA sequence; and
(ii) a mutation in the sequence that prevents a protein involved in regulating developmental timing, oncogenesis, and/or neuronal growth from blocking processing of the precursor microRNA sequence to a mature microRNA; and
(b) a DNA molecule encoding the precursor microRNA molecule of subparagraph (a).

12. The cell of claim 11, wherein the cell is a mammalian cell.

13. The cell of claim 12, wherein the cell is a human cell.

14. A method for making a precursor microRNA molecule such that the ability of a protein involved in regulating developmental timing, oncogenesis, and/or neuronal growth to decrease the level of the precursor microRNA molecule is blocked, the method comprising:
(a) providing a precursor microRNA sequence;
(b) making a mutation in the precursor microRNA sequence; and
wherein the ability of a protein involved in regulating developmental timing, oncogenesis, and/or neuronal growth to decrease the level of the microRNA molecule is blocked.

15. The method of claim 14, wherein the precursor microRNA sequence is Let-7 (SEQ ID NO: 9).

16. The method of claim 14, wherein the protein involved in regulating developmental timing, oncogenesis, and/or neuronal growth is Lin28.

17. The method of claim 14, wherein the mutation is in the part of the precursor microRNA sequence that forms a terminal loop structure.

18. The method of claim 17, wherein the mutation is a change from the nucleotide sequence GGAG to the nucleotide sequence GUAU.

19. The method of claim 14, wherein the mutation is made by performing PCR site-directed mutagenesis.

20. The method of claim 14 further comprising expressing the mutated precursor microRNA sequence in a cell.

21. The method of claim 20, wherein the cell is a mammalian cell.

22. The method of claim 21, wherein the cell is a human cell.

23. A method for elevating a level of a microRNA in a cell by making a precursor microRNA molecule, the method comprising:
(a) providing a precursor microRNA sequence;
(b) making a mutation in the precursor microRNA sequence such that the ability of a protein involved in regulating developmental timing, oncogenesis, and/or neuronal growth to decrease the levels of the microRNA molecule is blocked; and
(c) expressing the precursor microRNA molecule in a cell.

24. The method of claim 23, wherein the precursor microRNA molecule is Let-7 (SEQ ID NO: 12).

25. The method of claim 23, wherein the protein involved in regulating developmental timing, oncogenesis, and/or neuronal growth is Lin28.

26. The method of claim 23, wherein the mutation is in the part of the precursor microRNA sequence that forms a terminal loop structure.

27. The method of claim 23, wherein the mutation is a change from the nucleotides GGAG to the nucleotides GUAU.

28. The method of claim 23, wherein the mutation is made by performing PCR site-directed mutagenesis.

29. The method of claim 23, wherein the cell is a mammalian cell.

30. The method of claim 29, wherein the cell is a human cell.

31. The method of claim 23, wherein brain-derived neurotrophic factor (BDNF) translation specificity is inhibited.

32. The method of claim 23, wherein brain-derived neurotrophic factor (BDNF)-dependent dendrite arborization is inhibited.

33. The method of claim 23, wherein expressing the precursor microRNA sequence in a cell occurs with a vector comprising a DNA molecule encoding the precursor microRNA.

34. The method of claim 33, wherein the vector is a lentiviral vector.

35. A method for treating a cancer, the method comprising administering to a subject in need of treatment thereof, a therapeutically effective amount of a precursor microRNA comprising a mutation in the precursor microRNA sequence that prevents a protein involved in regulating developmental timing, oncogenesis, and/or neuronal growth from blocking processing of the precursor microRNA sequence to a mature microRNA.

36. The method of claim 35, wherein the precursor microRNA is Let-7 (SEQ ID NO: 12).

37. The method of claim 35, wherein the protein involved in regulating developmental timing, oncogenesis, and/or neuronal growth in Lin28.


SEQUENCE LISTING (45 sequences)

SEQ ID NO: 1 - DNA, 22 bp, Artificial Sequence
  Let-7a Northern blot probe
aactatacaacctactacctca

SEQ ID NO: 2 - DNA, 15 bp, Artificial Sequence
  Pre-Let-7a Northern blot probe
gtgggtgtgacccta

SEQ ID NO: 3 - DNA, 23 bp, Artificial Sequence
  miR-17 Northern blot probe
ctacctgcactgtaagcactttg

SEQ ID NO: 4 - DNA, 21 bp, Artificial Sequence
  shRNA targeting GW182 primer
ccttagtaatggagagtcaaa

SEQ ID NO: 5 - DNA, 25 bp, Artificial Sequence
  Let-7a-1 precursor miRNA forward outside primer for making GTAT mutation
tataggatcctcacacaggaaacca

SEQ ID NO: 6 - DNA, 25 bp, Artificial Sequence
  Let-7a-1 precursor miRNA reverse outside primer for making GTAT mutation
tatagctagcgctgcactacatctc

SEQ ID NO: 7 - DNA, 33 bp, Artificial Sequence
  Let-7a-1 precursor miRNA forward inside primer for making GTAT mutation
cccaccactggtatataactatacaatctactg

SEQ ID NO: 8 - DNA, 30 bp, Artificial Sequence
  Let-7a-1 precursor miRNA reverse inside primer to make GTAT mutation
tgtatagttatataccagtggtgggtgtga

SEQ ID NO: 9 - DNA, 78 bp, Homo sapiens
tgggatgaggtagtaggttgtatagttttaggtcacacccaccactgtatataactatacaatctactgtctttccta

SEQ ID NO: 10 - DNA, 78 bp, Artificial Sequence; identical to SEQ ID NO: 9
  Mutated DNA sequence encoding let-7a-1 miRNA

SEQ ID NO: 11 - RNA, 80 nt, Homo sapiens
ugggaugagguaguagguuguauaguuuuagggucacacccaccacugggagauaacuauacaaucuacugucuuuccua

SEQ ID NO: 12 - RNA, 78 nt, Artificial Sequence
  Mutated precursor let-7a-1 miRNA
ugggaugagguaguagguuguauaguuuuaggucacacccaccacuguauauaacuauacaaucuacugucuuuccua

SEQ ID NO: 13 - RNA, 23 nt, Homo sapiens
ccagugagccaggaacugcugcu

SEQ ID NO: 14 - RNA, 22 nt, Homo sapiens
gugccccacucacucugccucu

SEQ ID NO: 15 - RNA, 21 nt, Homo sapiens
ugccccacucacucugccucu

SEQ ID NO: 16 - RNA, 22 nt, Homo sapiens
gugccccacucacucugccucu

SEQ ID NO: 17 - RNA, 21 nt, Homo sapiens
uuaucuagacuuggcaucucu

SEQ ID NO: 18 - RNA, 22 nt, Homo sapiens
gagacuucacuacccuacuucc

SEQ ID NO: 19 - RNA, 22 nt, Homo sapiens
gagaccucacuacccuacuucc

SEQ ID NO: 20 - RNA, 21 nt, Homo sapiens
agaccucacuacccuacuucc

SEQ ID NO: 21 - RNA, 22 nt, Homo sapiens
gagaccucacuacccuacuucc

SEQ ID NO: 22 - RNA, 22 nt, Homo sapiens
cccgaaucauugugcuaucucu

SEQ ID NO: 23 - RNA, 21 nt, Homo sapiens
ccgaaucauugugcuaucucu

SEQ ID NO: 24 - RNA, 22 nt, Homo sapiens
cccgaaucauugugcuaucucu

SEQ ID NO: 25 - RNA, 22 nt, Homo sapiens
agaaacuuauuuuccuacuuca

SEQ ID NO: 26 - RNA, 21 nt, Homo sapiens
gaaacuuauuuuccuacuuca

SEQ ID NO: 27 - RNA, 22 nt, Homo sapiens
agaaacuuauuuuccuacuuca

SEQ ID NO: 28 - RNA, 22 nt, Homo sapiens
ucuauugugcccgccugccucc

SEQ ID NO: 29 - RNA, 21 nt, Homo sapiens
cuauugugcccgccugccucc

SEQ ID NO: 30 - RNA, 22 nt, Homo sapiens
ucuauugugcccgccugccucc

SEQ ID NO: 31 - RNA, 22 nt, Homo sapiens
uggcucuuguauggaaugcuga

SEQ ID NO: 32 - RNA, 22 nt, Homo sapiens
aacuuggacuuacguugcugcc

SEQ ID NO: 33 - RNA, 22 nt, Homo sapiens
uuggacuuacguugcugccucu

SEQ ID NO: 34 - RNA, 21 nt, Homo sapiens
uggucuuacguugcugccucu

SEQ ID NO: 35 - RNA, 22 nt, Homo sapiens
uuggacuuacguugcugccucu

SEQ ID NO: 36 - RNA, 23 nt, Homo sapiens
cgcuccacucucucaaauccugc

SEQ ID NO: 37 - RNA, 22 nt, Homo sapiens
gcugcccagggacaaguacagg

SEQ ID NO: 38 - RNA, 25 nt, Homo sapiens
agauggucacuaacucuaugcugca

SEQ ID NO: 39 - RNA, 21 nt, Homo sapiens
guaacugugaagagcaucuca

SEQ ID NO: 40 - RNA, 25 nt, Homo sapiens
aaaggggggaugggcagaguacagu

SEQ ID NO: 41 - RNA, 23 nt, Homo sapiens
cuggcauaagaggcaauguugcc

SEQ ID NO: 42 - RNA, 23 nt, Homo sapiens
ucuggcuuauucuccuacccucu

SEQ ID NO: 43 - RNA, 21 nt, Homo sapiens
cagcggacaaccaccaucucu

SEQ ID NO: 44 - RNA, 23 nt, Homo sapiens
gagaaagaaguuaccuuguauua

SEQ ID NO: 45 - RNA, 22 nt, Homo sapiens
agaaagaaguuaccuuguauua